(19) United States
(12) Patent Application Publication
(10) Pub. No.: US 2013/0232853 A1
(43) Pub. Date: Sep. 12, 2013

(54) METHOD FOR SELECTIVE PRODUCTION OF BIOBASED CHEMICALS AND BIOFUELS FROM PLANT LIGNIN

(71) Applicants: John R. Peterson, Chardon, OH (US); Jignesh S. Patel, (US); Vladimir Romakh, Mississauga (CA); Benjamin M.T. Scott, Guelph (CA)

(72) Inventors: John R. Peterson, Chardon, OH (US); Jignesh S. Patel, (US); Vladimir Romakh, Mississauga (CA); Benjamin M.T. Scott, Guelph (CA)

(73) Assignee: THESIS CHEMISTRY, LLC, Mentor, OH (US)

(21) Appl. No.: 13/470,398

(22) Filed: May 14, 2012

Related U.S. Application Data
(60) Provisional application No. 61/608,936, filed on Mar. 9, 2012.

(51) Int. Cl. C07G 1/00; C07C 253/00; C07C 67/00; C10G 1/00; C07C 63/331; C07C 45/00; C07C 41/01; C07C 37/00; C10L 1/02; C07C 63/16

(52) U.S. Cl. 44/307; 530/500; 530/505; 530/506; 530/507; 558/411; 558/424; 560/76; 560/129; 562/473; 562/480; 562/488; 568/426; 568/638; 568/643; 568/652; 568/716; 568/763; 585/240

(57) ABSTRACT

The present invention is directed generally to a method of production of biobased chemicals, biofuels, and lignin residues from lignin sources, including waste lignin. This method may allow for selectively producing biobased chemicals, biofuels, and lignin residues from lignin sources using certain processing methods. The methods for production of these biobased chemicals, biofuels, and lignin residues may be provided by chemical-induced processing, catalytic oxidative lignin depolymerisation processing, and catalytic hydroprocessing. Further, the catalytic hydroprocessing from processes including catalytic reduction processing, catalytic hydrodeoxygenation processing, and/or catalytic/dehydrogenation processing may also be used. The method described herein also provides a means in which waste from the process(es) may be reduced and/or recycled.

This application is a continuation-in-part and claims priority from U.S. Ser. No. 61/608,936, entitled A METHOD FOR PRODUCING BIOBASED CHEMICALS FROM PLANT LIGNIN, filed Mar. 9, 2012, which is incorporated herein by reference.

### I. BACKGROUND OF THE INVENTION

A. Field of Invention

The present invention is directed generally to a method of production of biobased chemicals, biofuels, and lignin residues from lignin sources, including waste lignin. A method for production may be provided by chemical-induced processing, catalytic oxidative lignin depolymerisation processing, and catalytic hydroprocessing. A method of selectively producing biobased chemicals, biofuels, and lignin residues from lignin sources is also described herein.

B. Description of the Related Art

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource. With the depletion of readily available oil reserves across the globe, the supply chain has shifted to more complex and environmentally risky production technologies. A reduction and conservation of fossil fuels is clearly needed. Some alternatives to fossil fuels, like solar power, wind power, geothermal power, hydropower, and nuclear power, are used to a degree. However, a more efficient use of renewable resources is always being sought.

As a stable and independent alternative to fossil fuels, biomass can be a potentially inexhaustible, domestic, natural resource for the production of energy, transportation fuels, and chemicals. The advantage in use of biomass for such purposes is magnified during an oil crisis, a surge in oil prices, or political unrest within oil producing regions of the world. Biomass includes plant and wood biomass, including agricultural biomass. Biomass can be employed as a sustainable source of energy and is a valuable alternative to fossil fuels. More specifically, the biorefining of biomass into derivative products typically produced from petroleum can help to stop the depletion of petroleum, or at least reduce the current demand and dependence. Biomass can become a key resource for chemical production in much of the world. Biomass, unlike petroleum, is renewable. Biomass can provide sustainable substitutes for petrochemically derived feedstocks used in existing markets.

Biomass is made up primarily of cellulose, hemicellulose, and lignin. These components, if economically separated from one another, can provide vital sources of chemicals normally derived from petrochemicals. The use of biomass can also be beneficial with agricultural and/or woody plants that are sparsely used and plant wastes that currently have little or no use. Biomass can provide valuable chemicals and reduce dependence on coal, gas, and fossil fuels, in addition to boosting local and worldwide economies.

In processes separating biomass, several options are available: the OrganoSolv™ and Alcell® processes which are used to efficiently remove the lignin from the other components under mild conditions, kraft pulping, sulfite pulping, pyrolysis, steam explosion, ammonia fiber explosion, dilute acid hydrolysis, alkaline hydrolysis, alkaline oxidative treatment, and enzymatic treatment. Kraft pulping is by far the dominant chemical pulping practiced in the world. However, often the removal of lignin from plant biomass can be a costly process, and some research efforts are now aimed at designing plants that either deposit less lignin or produce lignins that are more amenable to chemical degradation in order to avoid separating the biomass components.

Although the cellulosic fraction of biomass has garnered substantial attention recently as a feedstock for ethanol biofuel and other basic chemicals, the intrinsic value of the lignin continues to be largely overlooked. Lignin, which can comprise about 15% to about 30% of the organic matrix of woody and agricultural biomass, is the most abundant source of aromatic chemicals outside of crude oil. Lignin can be used in developing technologies that transform plant biomass into value-added aromatic chemicals.

Lignin has a complex, polymeric structure whose exact structure is unknown. This large group of aromatic polymers in lignin may be a result from the oxidative combinatorial linking of the 4-hydroxyphenyl propanoid building blocks provided by nature. The aromatic portion of these building blocks is composed of 4-hydroxyphenyl, guaiacyl (4-hydroxy-3-methoxyphenyl), and syringyl (4-hydroxyl-3,5-dimethoxyphenyl) units. These units may be abbreviated as H, G, and S, respectively. The lignin itself may also vary in the ratio of these units depending on its source.

Because of its make-up, lignin can be a source of aromatic chemicals outside of the conventional sources of petroleum and coal. Lignin may be obtained from wood and/or agricultural sources as fresh biomass. This wood and/or agricultural lignin may be waste lignin or recovered lignin from these sources. Lignin can also be obtained from multiple sources that utilize plant material, including pulp and paper mills and the sugar cane milling industries. It is also a major by-product in the cellulosic biomass-to-ethanol process. Often, these sources of lignin may be considered waste products where there can be an associated cost to dispose of the lignin instead of alternative methods where this lignin can provide value-added materials.

Another source of lignin is the black liquor produced from kraft pulp mills. In the kraft pulping process, lignin-rich black liquor is burnt in a recovery boiler to recover the spent alkali and to generate heat and power for mill operations. Some of the lignin in black liquor could be precipitated and used for value-added applications, especially since a production bottleneck may exist from the thermal capacity of the recovery boiler.

The present invention provides methods of production of biobased chemicals, biofuels, and lignin residues from lignin sources, including waste lignin, in which the end products may be selectively chosen. The present invention may also minimize waste products within the process described herein, providing a means in which waste from the process(es) may be reduced and/or recycled.

### II. SUMMARY OF THE INVENTION

Accordingly, it is an object of the present invention to provide a method for biorefining comprising the steps of providing lignin biomass; processing the lignin biomass; and producing at least one product from the lignin biomass.

One object of the present invention is that the lignin biomass is comprised of at least one lignin building block of p-coumaryl alcohol, coniferyl alcohol, and sinapyl alcohol.

According to another embodiment of the invention, the lignin biomass is provided from at least one biomass of plant biomass, woody plant biomass, agricultural plant biomass, and cultivated plant biomass.

According to another embodiment of the invention, the lignin biomass is provided from fresh plant biomass, recovered plant biomass, pulp and paper mill biomass, cellulosic ethanol refinery biomass, sugar cane mill biomass, commercial plant biomass fractionator biomass, and/or lignin processing residue biomass.

According to yet another embodiment of the invention, the lignin biomass is provided from kraft pulp mill lignin, sulfite pulp mill lignin, soda pulp mill lignin, cellulosic ethanol refinery lignin, and/or commercial plant biomass fractionator lignin.

Still another object of the present invention is that lignin biomass is provided from lignin residues lignin.

According to still another embodiment of the invention, the lignin biomass is provided from waste lignin.

One object of the present invention is that the waste lignin is provided by at least one waste lignin from recovered plant biomass waste lignin, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin.

One object of the present invention is that a lignin pretreatment is provided to the lignin biomass.

Another object of the present invention is that the processing of the lignin biomass is provided from at least one process of chemical-induced processing, catalytic oxidative lignin depolymerisation processing, and catalytic hydroprocessing.

Still another object of the present invention is that the chemical-induced processing is provided from at least one process of oxidative lignin depolymerisation processing and caustic-induced lignin depolymerisation processing.

Yet another object of the present invention is that the chemical-induced processing uses an oxidant.

Still yet another object of the present invention is that the oxidant comprises at least one oxidant of air, oxygen, hydrogen peroxide, hydrogen peroxide, organic peroxide, and organic nitro compound.

One object of the present invention is that the chemical-induced processing is controlled for selecting at least one of the products from the lignin biomass.

Another object of the present invention is that the chemical-induced processing is performed at a reaction temperature of about 50° C. to about 500° C.

Still another object of the present invention is that the chemical-induced processing is performed at a reaction temperature of about 80° C. to about 350° C.

Yet another object of the present invention is that the chemical-induced processing is performed at a reaction temperature of about 100° C. to about 250° C.

Still yet another object of the present invention is that the chemical-induced processing is induced by caustic.

Another object of the present invention is that the caustic is comprised of at least one caustic of lithium hydroxide, sodium hydroxide, potassium hydroxide, cesium hydroxide, magnesium hydroxide, barium hydroxide, calcium hydroxide, and carbonates and/or oxides of Group I and Group II metals of the Periodic Table.

Still another object of the present invention is that the chemical-induced processing is provided from a catalytic oxidative lignin depolymerisation process and/or a catalytic hydroprocessing process.

Yet another object of the present invention is that the chemical-induced processing can use an oxidant.

Still yet another object of the present invention is that the oxidant comprises at least one oxidant of air, oxygen, hydrogen peroxide, organic peroxide, and organic nitro compound.

One object of the present invention is that the chemical-induced processing is controlled for selecting at least one of the products from the lignin biomass.

Still another object of the present invention is that the catalytic oxidative lignin depolymerisation processing of the lignin biomass and at least one of the products of the lignin biomass is provided from at least one catalyst of a metal salt, a metal complex, and an elemental metal.

Yet another object of the present invention is that the catalyst used in the catalytic oxidative lignin depolymerisation processing is provided from at least one catalyst of the Group 3 through Group 12 transitional elements of the Periodic Table.

Still yet another object of the present invention is that the catalyst of the catalytic oxidative lignin depolymerisation processing is at least one catalyst type of a homogeneous catalyst, a heterogeneous catalyst, and a catalyst supported on an inert solid matrix.

Another object of the present invention is that the catalytic oxidative lignin depolymerisation processing provides non-selective oxidation of the lignin biomass or at least one of the products of the lignin biomass.

Still another object of the present invention is that the catalytic oxidative lignin depolymerisation processing provides selective oxidation of the lignin biomass and at least one of the products of the lignin biomass.

Yet another object of the present invention is that an oxidant is provided for the catalytic oxidative lignin depolymerisation processing and is selected from at least one oxidant of air, oxygen, hydrogen peroxide, hydrogen peroxide, organic peroxide, and organic nitro compound.

Still yet another object of the present invention is that the catalytic oxidative lignin depolymerisation processing is conducted at a reaction temperature of about 50° C. to about 300° C.

Another object of the present invention is that the catalytic oxidative lignin depolymerisation processing is conducted at a reaction temperature of about 100° C. to about 200° C.

Still another object of the present invention is that the catalytic oxidative lignin depolymerisation processing provides at least one of the products retaining at least 66% of the original carbon atom structure of the lignin biomass.

Yet another object of the present invention is that the catalytic oxidative lignin depolymerisation processing provides at least one of the products retaining at least 77% of the carbon atom structure of the lignin biomass.

Still yet another object of the present invention is that the catalytic oxidative lignin depolymerisation processing provides at least one of the products retaining at least 88% of the carbon atom structure of the lignin biomass.

Another object of the present invention is that the catalytic oxidative lignin depolymerisation processing provides at least one of the products retaining 100% of the carbon atom structure of the lignin biomass.

Still another object of the present invention is that the lignin biomass has a weight, and the catalytic oxidative lignin depolymerisation processing provides lignin residues having a weight of about 10% to about 90% of the lignin biomass weight.

Yet another object of the present invention is that the lignin biomass has a weight, and the catalytic oxidative lignin depolymerisation processing provides lignin residues having a weight of about 10% to about 50% of the lignin biomass weight.

Still yet another object of the present invention is that the catalytic hydroprocessing provides non-selective reduction of the lignin biomass and the products of the lignin biomass.

Another object of the present invention is that the catalytic hydroprocessing provides selective reduction of the lignin biomass and the products of the lignin biomass.

Still another object of the present invention is that the catalytic hydroprocessing of the lignin biomass and the products of the lignin biomass is provided by at least one process of catalytic reduction processing, catalytic hydrodeoxygenation processing, and catalytic hydrodeoxygenation/dehydrogenation processing.

Yet another object of the present invention is that the catalytic reduction processing, the catalytic hydrodeoxygenation processing, and the catalytic hydrodeoxygenation/dehydrogenation processing of the lignin biomass and the products of the lignin biomass is provided in any order.

Still yet another object of the present invention is that the catalytic reduction processing, the catalytic hydrodeoxygenation processing, and the catalytic hydrodeoxygenation/dehydrogenation processing of the lignin biomass and the products of the lignin biomass are provided by single stage processing or dual stage processing.

Another object of the present invention is that the catalytic hydrodeoxygenation/dehydrogenation processing further comprise the steps of processing using catalytic hydrodeoxygenation and processing using catalytic dehydrogenation.

Still another object of the present invention is that the catalytic hydrodeoxygenation processing and the catalytic hydrodeoxygenation/dehydrogenation processing further comprise the step of processing using catalytic dehydration.

Yet another object of the present invention is that the catalyst of the catalytic dehydration processing is provided by at least one catalyst of zeolite type catalysts, clay catalysts, and alumina support catalysts.

Still yet another object of the present invention is that the catalytic hydrodeoxygenation processing and catalytic hydrodeoxygenation/dehydrogenation processing provide at least one chemical of general molecular structure:

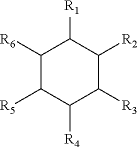

wherein R₁ is selected from among hydrogen, hydroxyl, and methoxy;
wherein R₂, R₃, R₄, R₅, and R₆ are selected from among hydrogen, methoxy, methyl, ethyl, propyl, isopropyl, butyl, isobutyl, and t-butyl; and
wherein unsaturation can exist in at least one of the products of the catalytic hydrodeoxygenation processing.

Still another object of the present invention is that the catalytic hydrodeoxygenation and hydrodeoxygenation/dehydrogenation processing provides at least one chemical of general molecular structure:

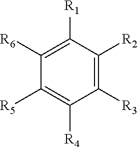

wherein R₁ is selected from among hydrogen, hydroxyl, and methoxy; and
wherein R₂, R₃, R₄, R₅, and R₆ are selected from among hydrogen, methoxy, methyl, ethyl, propyl, isopropyl, butyl, isobutyl, and t-butyl.

Yet another object of the present invention is that the catalytic hydroprocessing of the lignin biomass and at least one of the products of the lignin biomass is performed at a reaction temperature of about 50° C. to about 500° C.

Still yet another object of the present invention is that the catalytic hydroprocessing of said lignin biomass and at least one of the products of the lignin biomass is performed at a reaction temperature of about 50° C. to about 300° C.

Another object of the present invention is that the catalytic hydroprocessing uses at least one catalyst provided from the Group 3 through Group 12 transitional elements of the Periodic Table.

Still another object of the present invention is that the catalytic hydroprocessing uses at least one catalyst provided from the Group III through Group V elements of the Periodic Table.

Yet another object of the present invention is that the catalytic hydroprocessing uses a reducing agent provided by at least one reducing agent of hydrogen and hydrogen-donating liquids.

Still yet another object of the present invention is that the processing of the lignin biomass is provided from at least one process of batch processing and flow processing.

Another object of the present invention is that the processing of the lignin biomass is conducted in caustic provided by at least one caustic of lithium hydroxide, sodium hydroxide, potassium hydroxide, cesium hydroxide, magnesium hydroxide, barium hydroxide, and calcium hydroxide.

Still another object of the present invention is that the processing of the lignin biomass is conducted in solvent provided by at least one solvent of water, ethanol, propanol, isopropanol, acetonitrile, and ionic liquids.

Yet another object of the present invention is that at least one of the products from the lignin biomass comprises at least one product of biobased chemicals, biofuels, and lignin residues.

Still yet another object of the present invention is that at least one of the products from the lignin biomass comprises at least two products of biobased chemicals, biofuels, and lignin residues.

Another object of the present invention is that the biobased chemicals comprise at least one chemical of commodity chemicals, fine chemicals, and specialty chemicals.

Still another object of the present invention is that the biobased chemicals comprise at least one chemical of achiral chemicals, racemic chemicals, and chiral chemicals.

Yet another object of the present invention is that a ratio of the p-coumaryl alcohol, coniferyl alcohol, and sinapyl alcohol lignin building blocks provides control of a composition of at least one of the products from the lignin biomass.

Still yet another object of the present invention is that the ratio of the p-coumaryl alcohol, coniferyl alcohol, and sinapyl alcohol lignin building blocks provides control of a composition of at least two of the products from the lignin biomass.

Another object of the present invention is that the biobased chemicals comprise at least one chemical of aryl aldehydes, aryl carboxylic acids, aryl ketones, and aliphatic carboxylic acids.

Still another object of the present invention is that the biobased chemicals comprise at least two chemicals of aryl aldehydes, aryl carboxylic acids, aryl ketones, and aliphatic carboxylic acids.

Yet another object of the present invention is that at least one chemical of aryl aldehydes, aryl carboxylic acids, aryl ketones, and aliphatic carboxylic acids are provided by catalytic oxidative lignin depolymerisation processing.

Still yet another object of the present invention is that aryl aldehydes comprise at least one chemical of 4-hydroxybenzaldehyde, vanillin, and syringaldehyde.

Another object of the present invention is that aryl aldehydes comprise at least one chemical of (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, and 4-hydroxy-3,5-dimethoxycinnaminaldehyde.

Still another object of the present invention is that aryl carboxylic acids comprise at least one chemical of 4-hydroxybenzoic acid, vanillic acid, and syringic acid.

Yet another object of the present invention is that aryl carboxylic acids comprise at least one chemical of general molecular structure:

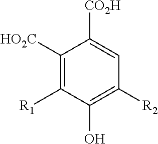

wherein R₁ and R₂ are selected from among hydrogen and methoxy.

Still yet another object of the present invention is that aryl carboxylic acids comprise at least one chemical of general molecular structure:

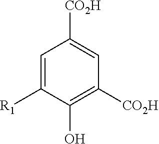

wherein R₁ is selected from among hydrogen and methoxy.

Another object of the present invention is that aryl carboxylic acids comprise at least one chemical of general molecular structure:

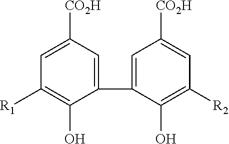

wherein R₁ and R₂ are selected from among hydrogen and methoxy.

Still another object of the present invention is that aryl carboxylic acids comprise at least one chemical of general molecular structure:

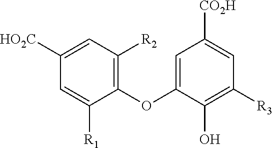

wherein R₁, R₂, and R₃ are selected from among hydrogen and methoxy.

Yet another object of the present invention is that aryl carboxylic acids comprise at least one chemical of (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

Still yet another object of the present invention is that aryl aldehydes and the aryl carboxylic acids comprise at least one chemical of 4-hydroxybenzaldehyde, vanillin, syringaldehyde, 4-hydroxybenzoic acid, vanillic acid, and syringic acid.

Another object of the present invention is that aryl ketones comprise at least one chemical of 1-(4-hydroxyphenyl)ethanone, 1-(4-hydroxy-3-methoxyphenyl)ethanone, and 1-(4-hydroxy-3,5-dimethoxyphenyl)ethanone.

Still another object of the present invention is that aryl ketones comprise at least one chemical of 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1-propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, and 1-(4-hydroxy-3,5-dimethoxyphenyl)-2-propanone.

Yet another object of the present invention is that aliphatic carboxylic acids comprise at least one chemical of formic acid, oxalic acid, acetic acid, glycolic acid, glyoxylic acid, propionic acid, lactic acid, and malonic acid.

Still yet another object of the present invention is that biobased chemicals comprise at least one chemical of phenols, alkyl phenols, alkenyl phenols, and performance chemicals.

Another object of the present invention is that biobased chemicals comprise at least two chemicals of phenols, alkyl phenols, alkenyl phenols, and performance chemicals.

Still another object of the present invention is that at least one chemical of phenols, alkyl phenols, alkenyl phenols, and performance chemicals are provided by catalytic hydroprocessing.

Yet another object of the present invention is that phenols comprise at least one chemical of phenol, guaiacol, and 2,6-dimethoxyphenol.

Still yet another object of the present invention is that alkyl phenols comprise at least one chemical of 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, and 2,4,6-trimethylphenol.

Another object of the present invention is that alkyl phenols comprise at least one chemical of a general molecular structure:

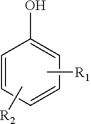

wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₁ and R₂ are located at positions 2, 3, 4, or 5 of the phenol ring.

Still another object of the present invention is that alkyl phenols comprise at least one chemical of a general molecular structure:

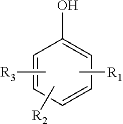

wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

Yet another object of the present invention is that alkyl phenols comprise at least one chemical of 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, and 2,6-dimethoxy-4-t-butylphenol.

Still yet another object of the present invention is that alkyl phenols comprise at least one chemical of general molecular structure:

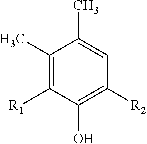

wherein R₁ and R₂ are selected from among hydrogen and methoxy.

Another object of the present invention is that alkyl phenols comprise at least one chemical of general molecular structure:

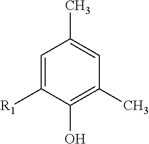

wherein R₁ is selected from among hydrogen and methoxy.

Still another object of the present invention is that alkyl phenols comprise at least one chemical of general molecular structure:

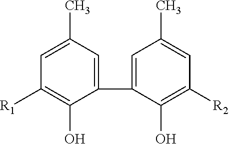

wherein R₁ and R₂ are selected from among hydrogen and methoxy.

Yet another object of the present invention is that alkyl phenols comprise at least one chemical of general molecular structure:

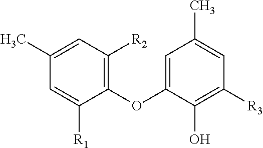

wherein R₁, R₂, and R₃ are selected from among hydrogen and methoxy.

Still yet another object of the present invention is that alkenyl phenols comprise at least one chemical of 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and iso-syringeugenol.

Another object of the present invention is that performance chemicals comprise at least one chemical of products comprising phenols, alkyl phenols, and alkenyl phenols.

Still another object of the present invention is that the biobased chemicals comprise at least one chemical of benzene, toluene, xylenes, mesitylenes, biaryls, aryl alkanes, aryl alkenes, alkanes, alkenes, cycloalkanes, cycloalkenes, alkyl esters, and performance chemicals.

Yet another object of the present invention is that the biobased chemicals comprise at least two chemicals of benzene, toluene, xylenes, mesitylenes, biaryls, aryl alkanes, aryl alkenes, alkanes, alkenes, cycloalkanes, cycloalkenes, alkyl esters, and performance chemicals.

Yet another object of the present invention is that at least one chemical of benzene, toluene, xylenes, mesitylenes, biaryls, aryl alkanes, aryl alkenes, alkanes, alkenes, cycloalkanes, cycloalkenes, alkyl esters, and performance chemicals are provided by hydroprocessing processing.

Still yet another object of the present invention is that the biobased chemicals comprise at least one chemical of benzene, toluene, 1,2-dimethylbenzene, 1,3-dimethylbenzene, 1,4-dimethylbenzene, 1,2,3-trimethylbenzene, 1,2,4-trimethylbenzene and 1,3,5-trimethylbenzene.

Another object of the present invention is that biaryls comprise at least one chemical of biphenyl, 4,4′-dimethylbiphenyl, 3,3′-dimethylbiphenyl, 2,2′-dimethylbiphenyl, 3,4′-dimethylbiphenyl, 2,4′-dimethylbiphenyl, 2,3′-dimethylbiphenyl, 4,4′-diethylbiphenyl, 3,3′-diethylbiphenyl, 2,2′-diethylbiphenyl, 3,4′-diethylbiphenyl, 2,4′-diethylbiphenyl, 2,3′-diethylbiphenyl, 4,4′-dipropylbiphenyl, 3,3′-dipropylbiphenyl, 2,2′-dipropylbiphenyl, 3,4′-dipropylbiphenyl, 2,4′-dipropylbiphenyl, and 2,3′-dipropylbiphenyl.

Still another object of the present invention is that aryl alkanes comprise at least one chemical of ethylbenzene, propylbenzene, isopropylbenzene, butylbenzene, isobutylbenzene, and t-butylbenzene.

Yet another object of the present invention is that aryl alkanes comprise at least one chemical of a general molecular structure:

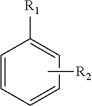

wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₂ is located at positions 2, 3, 4, or 5 of the ring.

Still yet another object of the present invention is that aryl alkanes comprise at least one chemical of a general molecular structure:

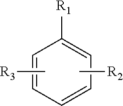

wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and t-butyl; and
wherein R₂ and R₃ are located at positions 2, 3, 4, or 5 of the ring.

Another object of the present invention is that aryl alkenes comprise at least one chemical of styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, and 1-(4-methylphenyl)-2-propene.

Still another object of the present invention is that alkanes comprise at least one chemical of hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, and 3,4,5-trimethylnonane.

Yet another object of the present invention is that alkenes comprise at least one compound of a partially unsaturated alkane.

Still yet another object of the present invention is that cycloalkanes comprise at least one chemical of cyclopentane, cyclohexane, cycloheptane, methylcyclopentane, methylcyclohexane, methylcycloheptane, ethylcyclopentane, ethylcyclohexane, ethylcycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and 1,4-dimethylcycloheptane.

Another object of the present invention is that cycloalkanes comprise at least one chemical of a general molecular structure:

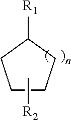

wherein n is 1, 2, or 3;
wherein R₁ is selected from among methyl, ethyl, propyl, isopropyl, butyl, isobutyl, and t-butyl;
wherein R₂ is selected from among ethyl, propyl, propyl, isopropyl, butyl, isobutyl, and t-butyl; and
wherein R₂ is located at any ring position other than that of R₁.

Still another object of the present invention is that cycloalkenes comprise at least one compound of a partially unsaturated cycloalkane.

Yet another object of the present invention is that alkyl esters comprise at least one chemical of a general molecular structure:

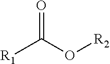

wherein R₁ and R₂ are selected from among methyl, ethyl, propyl, isopropyl, butyl, isobutyl, and t-butyl.

Yet another object of the present invention is that performance chemicals comprise at least one chemical of benzene, toluene, xylenes, mesitylenes, aryl alkanes, aryl alkenes, alkanes, alkenes, cycloalkanes, cycloalkenes, and alkyl esters.

Still yet another object of the present invention is that biofuels comprise at least one chemical of alkanes, alkenes, cycloalkanes, cycloalkenes, alkyl esters, benzene, toluene, xylenes, mesitylenes, biaryls, aryl alkanes, aryl alkenes, alkyl naphthalenes, phenols, alkyl phenols, and alkenyl phenols.

Another object of the present invention is that biofuels comprise blends of at least two chemicals of alkanes, alkenes, cycloalkanes, cycloalkenes, alkyl esters, benzene, toluene, xylenes, mesitylenes, biaryls, aryl alkanes, aryl alkenes, alkyl naphthalenes, phenols, alkyl phenols, and alkenyl phenols.

Still another object of the present invention is that blends of the biofuels comprise product mixtures of chemicals of similar boiling point range.

Yet another object of the present invention is that blends of the biofuels comprise product mixtures of chemicals with a carbon and hydrogen content of about 80% to about 100%.

Still yet another object of the present invention is that blends of the biofuels comprise product mixtures of chemicals with a research octane number of at least about 90.

Another object of the present invention is that blends of the biofuels are comprised of at least one fuel of transportation fuels, heating fuels, and fuel additives.

Still another object of the present invention is that transportation fuels serve at least one market of automobile fuels, truck fuels, ship fuels, and aircraft fuels.

Yet another object of the present invention is that heating fuels serve at least one market of home heating fuels, commercial heating fuels, and industrial boiler fuels.

Still yet another object of the present invention is that fuel additives serve at least one market of transportation fuels and heating fuels.

Another object of the present invention is that the process further comprises the step of using at least one product from the lignin biomass in the production of other derivative chemicals, materials, and products.

Still another object of the present invention is that other derivative chemicals, materials, and products comprise at least one chemical of aryl aldehydes, aryl carboxylic acids, aryl nitriles, aryl alcohols, and aryl esters.

Yet another object of the present invention is that aryl aldehydes of the derivative chemicals, materials, and products comprise at least one chemical of 4-hydroxybenzaldehyde, vanillin, and syringaldehyde.

Yet another object of the present invention is that aryl aldehydes of the derivative chemicals, materials, and products comprise at least one chemical of general molecular structure:

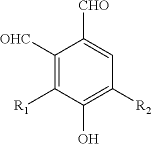

wherein R₁ and R₂ are selected from among hydrogen and methoxy.

Still yet another object of the present invention is that aryl aldehydes of the derivative chemicals, materials, and products comprise at least one chemical of general molecular structure:

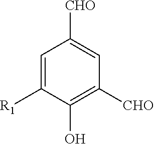

wherein R₁ is selected from among hydrogen and methoxy.

Another object of the present invention is that aryl aldehydes of the derivative chemicals, materials, and products comprise at least one chemical of general molecular structure:

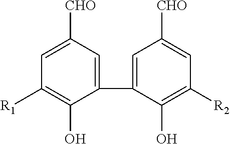

wherein R₁ and R₂ are selected from among hydrogen and methoxy.

Still another object of the present invention is that aryl aldehydes of the derivative chemicals, materials, and products comprise at least one chemical of general molecular structure:

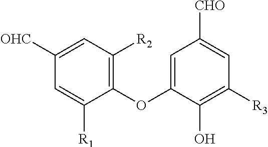

wherein R₁, R₂, and R₃ are selected from among hydrogen and methoxy.

Yet another object of the present invention is that aryl carboxylic acids of the derivative chemicals, materials, and products comprise at least one chemical of 4-hydroxybenzoic acid, vanillic acid, and syringic acid.

Still yet another object of the present invention is that aryl nitriles of the derivative chemicals, materials, and products comprise at least one chemical of 4-hydroxybenzonitrile, 4-hydroxy-3-methoxybenzonitrile, and 4-hydroxy-3,5-dimethoxybenzonitrile.

Another object of the present invention is that aryl nitriles of the derivative chemicals, materials, and products comprise at least one chemical of general molecular structure:

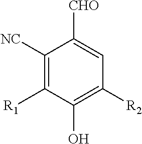

wherein R₁ and R₂ are selected from among hydrogen and methoxy.

Still another object of the present invention is that aryl nitriles of the derivative chemicals, materials, and products comprise at least one chemical of general molecular structure:

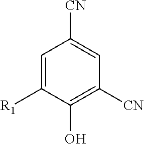

wherein R₁ is selected from among hydrogen and methoxy.

Yet another object of the present invention is that aryl nitriles of the derivative chemicals, materials, and products comprise at least one chemical of general molecular structure:

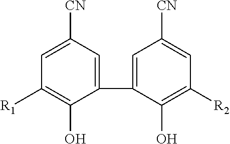

wherein R₁ and R₂ are selected from among hydrogen and methoxy.

Yet another object of the present invention is that aryl nitriles of the derivative chemicals, materials, and products comprise at least one chemical of general molecular structure:

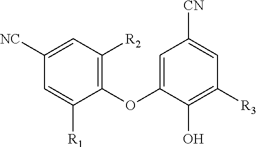

wherein R₁, R₂, and R₃ are selected from among hydrogen and methoxy.

Still yet another object of the present invention is that aryl alcohols of the derivative chemicals, materials, and products comprise at least one chemical of 4-hydroxybenzyl alcohol, 4-hydroxy-3-methoxybenzyl alcohol, and 4-hydroxy-3,5-dimethoxybenzyl alcohol.

Another object of the present invention is that aryl alcohols of the derivative chemicals, materials, and products comprise at least one chemical of general molecular structure:

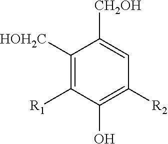

wherein R₁ and R₂ are selected from among hydrogen and methoxy.

Still another object of the present invention is that aryl alcohols of the derivative chemicals, materials, and products comprise at least one chemical of general molecular structure:

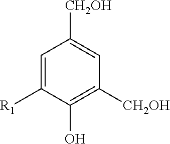

wherein R₁ is selected from among hydrogen and methoxy.

Yet another object of the present invention is that aryl alcohols of the derivative chemicals, materials, and products comprise at least one chemical of general molecular structure:

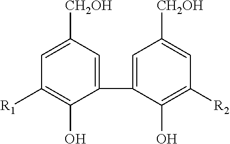

wherein R₁ and R₂ are selected from among hydrogen and methoxy.

Another object of the present invention is that aryl alcohols of the derivative chemicals, materials, and products comprise at least one chemical of general molecular structure:

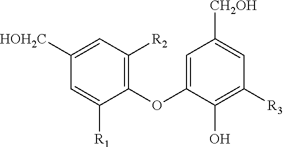

wherein R₁, R₂, and R₃ are selected from among hydrogen and methoxy.

Still yet another object of the present invention is that aryl esters of the derivative chemicals, materials, and products comprise a C₁-C₁₆ ester of at least one chemical of 4-hydroxybenzoic acid, vanillic acid, and syringic acid.

Yet another object of the present invention is that aryl esters of the derivative chemicals, materials, and products comprise a C₁-C₁₆ ester of at least one chemical of general molecular structure:

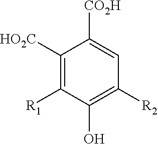

wherein R₁ and R₂ are selected from among hydrogen and methoxy.

Still yet another object of the present invention is that aryl esters of the derivative chemicals, materials, and products comprise a C₁-C₁₆ ester of at least one chemical of general molecular structure:

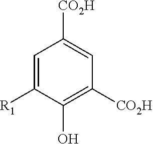

wherein R₁ is selected from among hydrogen and methoxy.

Another object of the present invention is that aryl esters of the derivative chemicals, materials, and products comprise a C₁-C₁₆ ester of least one chemical of general molecular structure:

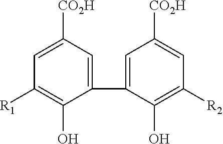

wherein R₁ and R₂ are selected from among hydrogen and methoxy.

Still another object of the present invention is that aryl esters of the derivative chemicals, materials, and products comprise a C₁-C₁₆ ester of at least one chemical of general molecular structure:

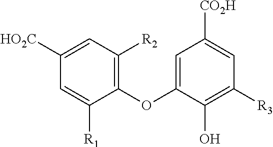

wherein R₁, R₂, and R₃ are selected from among hydrogen and methoxy.

Still another object of the present invention is that lignin residues provide energy production.

Yet another object of the present invention is that energy production from lignin residue is heat or power.

Still yet another object of the present invention is that lignin residue is subjected to further processing to produce at least one additional product.

Another object of the present invention is that lignin biomass has a weight, and a waste product of the lignin biomass is less than 30% of the lignin biomass weight.

Still another object of the present invention is that lignin biomass has a weight, and a waste product of the lignin biomass is less than 20% of the lignin biomass weight.

Yet another object of the present invention is that lignin biomass has a weight, and a waste product of the lignin biomass is less than 10% of the lignin biomass weight.

Yet another object of the present invention is that waste products from processing of the lignin biomass provide energy production.

Still yet another object of the present invention is that energy production from the waste products provide heat or power.

Another object of the present invention is that the process further comprises the step of recovering and recycling caustic from the processing of the lignin biomass.

Still another object of the present invention is that size exclusion membrane filtration is used for recovering and recycling caustic from the processing of the lignin biomass.

Yet another object of the present invention is that a pH precipitation is used for the recovering and recycling caustic from the processing of lignin biomass.

Still yet another object of the present invention is that the process described herein further comprises the step of functionalizing the lignin biomass prior to producing at least one of the products from the lignin biomass.

Another object of the present invention is that the product of the lignin biomass has an economic value higher than boiler fuel.

Still another object of the present invention is that the processing of lignin biomass produces at least two products of differing economic value.

Yet another object of the present invention is that the process allows for selective production of a product from the lignin biomass.

Still yet another object of the present invention is that it provides a method for biorefining, comprising the steps of: providing lignin biomass comprising at least one biomass of woody plant biomass, agricultural plant biomass, cultivated plant biomass, kraft pulping biomass, sulfite pulping biomass, soda pulping biomass, cellulosic ethanol refinery biomass, sugar cane mill biomass, lignin residue biomass, and waste biomass; processing the lignin biomass with chemical-induced lignin depolymerisation processing, catalytic oxidative lignin depolymerisation processing, and catalytic hydroprocessing; processing the lignin biomass with catalytic hydroprocessing from at least one process of catalytic reduction processing, catalytic hydrodeoxygenation processing, and catalytic hydrodeoxygenation/dehydrogenation processing; processing of the lignin biomass from at least one catalytic process to selectively provide at least one product which retains at least 77% of the carbon atom structure of the lignin biomass; functionalizing the lignin biomass prior to producing at least one product from the lignin biomass; producing at least one product from the lignin biomass comprising at least one product of biobased chemicals, biobased fuels, and lignin residues; producing a plurality of products from the lignin biomass comprising at least one chemical of aryl aldehydes, aryl carboxylic acids, aryl ketones, aliphatic carboxylic acids, phenols, alkyl phenols, alkenyl phenols, benzene, toluene, xylenes, mesitylenes, biaryls, aryl alkanes, aryl alkenes, alkanes, alkenes, cycloalkanes, cycloalkenes, alkyl esters, and performance chemicals; reducing the waste product of the lignin biomass, wherein the lignin biomass has a weight, and the waste product of the lignin biomass is less than 20% of the lignin biomass weight; producing energy utilizing the lignin residues; producing energy utilizing the waste product of the lignin biomass biomass; recovering and recycling caustic from the processing of the lignin; and using at least one product from the lignin biomass in the production of other derivative chemicals, materials, and products; wherein choosing of a source of the lignin biomass provides a selective production of at least one of the products from the lignin biomass.

Another object of the present invention is that the product distribution from the process described herein parallels the H:G:S building block ratio of the lignin itself.

Still another object of the present invention is that the selection of the lignin source can therefore allow for the prediction of a certain product ratio.

Further, another object of the present invention can be to provide a method for biorefining that is easy to implement and use.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, and wherein:

FIG. 1 is a flow diagram schematically illustrating lignin sources in the present invention.

FIG. 2 is a schematic illustrating the building blocks of lignin.

FIG. 3 is a table illustrating the structural linkages of lignin.

FIG. 4 is a schematic illustrating the present invention.

FIG. 5 is a flow diagram schematically illustrating another aspect of the present invention.

FIG. 6 is a flow diagram schematically illustrating another aspect of the present invention.

FIG. 7 is a flow diagram schematically illustrating another aspect of the present invention.

FIG. 8 is a series of schematics illustrating another aspect of the present invention.

FIG. 9 is a flow diagram schematically illustrating another aspect of the present invention.

FIG. 10 is a flow diagram schematically illustrating another aspect of the present invention.

FIG. 11 is a flow diagram schematically illustrating another aspect of the present invention.

FIG. 12 is a flow diagram schematically illustrating another aspect of the present invention.

FIG. 13 is a flow diagram schematically illustrating another aspect of the present invention.

FIG. 14 is a flow diagram schematically illustrating another aspect of the present invention.

FIG. 15 is a flow diagram schematically illustrating another aspect of the present invention.

### IV. DETAILED DESCRIPTION OF THE INVENTION

Referring now to the drawings wherein the showings are for purposes of illustrating embodiments of the invention only and not for purposes of limiting the same.

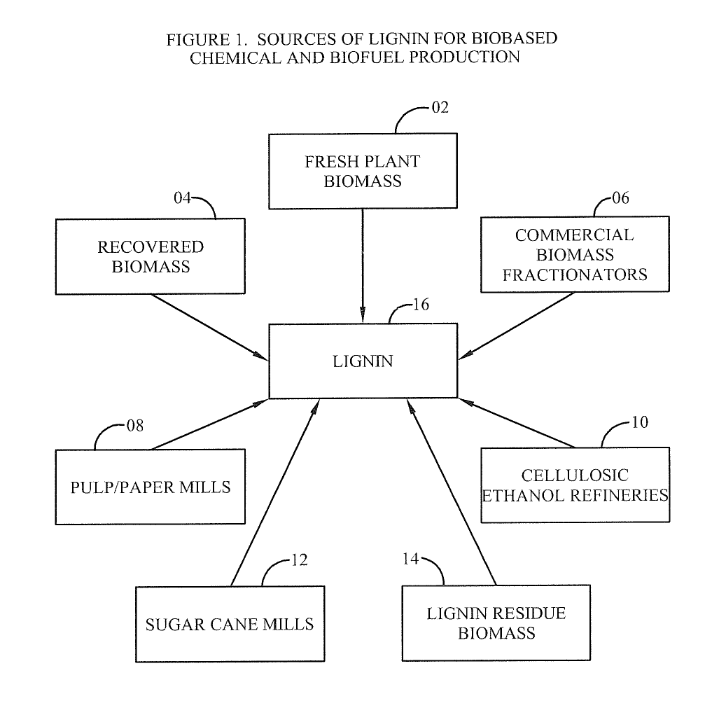

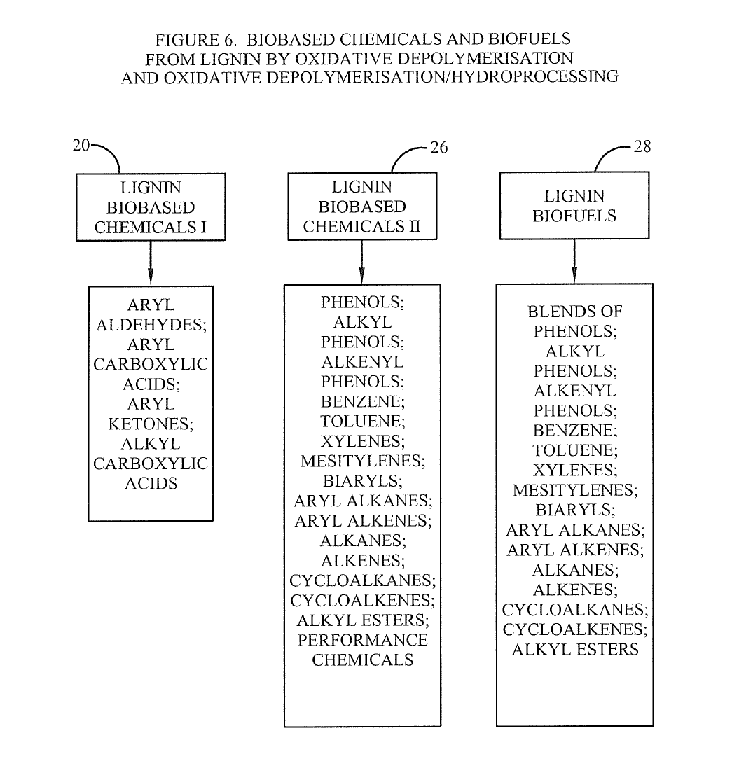

FIG. 1 provides a schematic overview where lignin 16 may be provided from various sources. The sources for the lignin 16 may include fresh plant biomass 2, recovered biomass 4, commercial biomass fractionators 6, pulp and paper mills 8, cellulosic ethanol refineries 10, sugar can mills 12, and/or lignin residue biomass 14. In processing the lignin 16, it may be converted into other chemical-based products, as shown in FIG. 6.

Lignin 16 may be the most abundant source of aromatic chemicals outside of crude oil and coal. Lignin 16 can be used in developing technologies that transform various sources of biomass and lignin 16 waste into value-added aromatic chemicals. The sources of lignin 16 may include at least one biomass of plant biomass, woody plant biomass, agricultural plant biomass, and cultivated plant biomass. The sources of lignin 16 may include fresh plant biomass 2, recovered biomass 4, commercial biomass fractionators 6, pulp and paper mills 8, cellulosic ethanol refineries 10, sugar can mills 12, and/or lignin residue biomass 14. Although these sources of lignin 16 can be used, these sources of lignin 16 are not limited to only those listed herein. No matter the origin of the lignin 16, any different sources of lignin 16 may be used within the process described herein.

Lignin 16 may be a structurally complex, polymeric substance made up of 4-hydroxyphenyl propanoid building blocks containing 4-hydroxyphenyl (abbreviated as H), guaiacyl (4-hydroxy-3-methoxyphenyl) (abbreviated as G), and syringyl (4-hydroxy-3,5-dimethoxyphenyll) units (abbreviated as S). The abundance of each of these units within the lignin 16 may change somewhat between individual plant species for woody lignin, namely lignin content for hardwoods and softwoods, as well as for agricultural sources and both cultivated and uncultivated plants. This difference in the units based on the species for the lignin 16 can control, or at least predict, the amounts and types of chemical products that may be produced within the process described herein.

To begin the process described herein, fresh plant biomass 2 may be utilized as a lignin source. Fresh plant biomass 2 may be considered to be biomass from agricultural plants, woody plants, and/or other plant biomass sources. Fresh plant biomass 2 may also include cultivated plant biomass. Fresh plant biomass 2 may be used where it may be grown specifically for this application, which may include, but is not limited to, switchgrass, miscanthus, hybrid eucalyptus trees, and hybrid poplar trees. Some fresh plant biomass 2 not specifically grown for this application may include agricultural or tree harvesting surplus. Where fresh plant biomass 2 is used, the lignin 16 can be separated from the other components like cellulose, hemicellulose, and other extractives. After the lignin 16 is separated, it may be added to the process described herein.

Sources of recovered biomass 4 may include several biomass waste products. The recovered biomass 4 can include woody biomass like wood chips, sawdust, and/or recovered wood, and/or agricultural plant biomass like wheat straw, rice straw, corn stover and/or other agricultural products typically left to rot in the field. Additionally, other plant biomass may also include lawn and tree maintenance byproducts. Another potential source of lignin 16 from recovered biomass 4 may include sugar cane milling. Sugar cane milling may provide lignin 16 since bagasse, or sugar cane waste fiber, can be generated. Bagasse is the name given to the discarded husks of the sugar cane plant after they have been pressed to extract the juices which are refined to make sugar. This agricultural waste can be very plentiful and may otherwise be burnt or discarded in the sugar cane milling process. Recovered biomass 4 may also include other waste products, including at least one waste lignin of sulfite pulping mill waste lignin, kraft pulping mill waste lignin, soda pulping mill waste lignin, and sugar cane mill waste lignin.

Both the fresh plant biomass 2 and the recovered biomass 4 may be treated to provide lignin 16 using any of the methods described in U.S. utility applications: A METHOD FOR PRODUCING BIOBASED CHEMICALS FROM PLANT BIOMASS (U.S. application Ser. No. 13/292,222 filed Nov. 9, 2011), A METHOD FOR PRODUCING BIOBASED CHEMICALS FROM WOODY BIOMASS (U.S. application Ser. No. 13/292,437 filed Nov. 9, 2011), A METHOD FOR PRODUCING BIOBASED CHEMICALS FROM AGRICULTURAL BIOMASS (U.S. application Ser. No. 13/292,531 filed Nov. 9, 2011), and A METHOD FOR PRODUCING BIOBASED CHEMICALS FROM CULTIVATED PLANT BIOMASS (U.S. application Ser. No. 13/292,632 filed Nov. 9, 2011).

Another source of lignin 16 may be commercial biomass fractionators 6. These commercial biomass fractionators 6 can be a thermal and/or mechanical processor which directly inputs raw biomass such as fresh plant biomass 2, woodchips and crop waste and produces multiple component streams, which may include sugars, cellulose, hemicellulose, and lignin 16. One example of a commercial biomass fractionator 6 may be Vertichem Corporation. Some of these component streams may include lignin 16 streams to produce useful products such as aryl aldehydes, aryl carboxylic acids, aryl esters, aryl ketones, aryl alcohols, aliphatic carboxylic acids, phenols, alkyl phenols, alkenyl phenols, benzene, toluene, xylene (collectively, benzene, toluene, and xylene are often referred to as “BTX”), mesitylenes, biaryls, aryl alkanes, aryl alkenes, alkanes, alkenes, cycloalkanes, cycloalkenes, alkyl esters, and performance chemicals. Within the process, the biomass may be treated to yield a highly pure cellulose fraction. Several different methods may be used for the separation, including pH, temperature, and pressure adjustments. A reaction involving enzymes may also be used. Other methods of fractionation may include chemical, mechanical, and biological methods. For instance, the biomass fractionator may separate the cellulose out by hot water treatments, hot alkaline treatments, and/or an alkaline oxidation step. Although the commercial biomass fractionators 6 may provide useful biobased products, they may also produce or leave behind other solids comprising of lignin 16. Instead of becoming a waste product, these lignin 16 solids may be used within the process described herein.

Pulp and paper mills 8 may also contribute to the lignin 16 from kraft pulping, sulfite pulping, and soda pulping. Lignin 16 can be removed during paper processing in a pulp and paper mills 8, where it is typically viewed as an undesirable component of biomass that requires both energy and chemicals to remove it during the pulping operation. These pulp and paper mills 8 may generally recover the lignin 16 as a by-product of the pulping process and may use it as boiler fuel. This removal of lignin 16 may be done by a chemical removal, with or without mechanical means. Some chemical methods of lignin 16 removal from pulp and paper mills 8 may be kraft pulping, sulfite pulping, and soda pulping.

The more dominant chemical pulping technique employed can be kraft processing, which employs high pHs by using considerable amounts of aqueous sodium hydroxide and sodium sulfide at high temperatures to degrade cellulosic biomass into cellulose, hemicellulose, and lignin 16 in a stepwise process. In the kraft process, black liquor can be burnt in a recovery boiler to recover the spent alkali and to generate heat and power for mill operations. However, some of the lignin 16 in black liquor can be precipitated and used for value-added applications where these exist. This conversion to value-added applications may be particularly attractive for a kraft pulping mill where a production bottleneck exists due to the thermal capacity of the recovery boiler. This process may provide kraft lignin.

The sulfite processing yielding lignosulfonates can also be relatively common in the pulp and paper industry. The sulfite process may be conducted between about pH 2 to about pH 12 using sulfite with a counterion. This counterion may be either calcium or magnesium. The product may be soluble in water as well as some highly polar organics and amines.

The soda pulp mill may also provide another chemical pulping process where caustic soda can be used to produce pulp. Although it is an old method, it can be effective in separating pulp from wood and grasses.

Another source of lignin 16 may also be cellulosic ethanol refineries 10. With the cellulosic ethanol refineries 10, they may produce lignin 16 and other by-products in the cellulosic biomass-to-ethanol process, which can also be used to produce energy required for the ethanol production process. Cellulosic ethanol refineries 10 produce ethanol fuel. The cellulosic ethanol can be made from plant materials like miscanthus, switchgrass, wheat stalks, corn stover, and woody biomass.

Cellulosic ethanol refineries 10 may use the OrganoSolv™ process or the Alcell® process to obtain lignin 16. OrganoSolv™ lignin may be obtained by treatment of fresh plant biomass 2 or bagasse, the fibrous residue that remains after plant material may be treated with various organic solvents. The OrganoSolv™ process may produce separate streams of cellulose, hemicelluloses, and lignin 16. It can be considered environmentally friendly because it may not use the sulfides, sulfites, and harsh conditions used in the kraft or lignosulfonate pulping processes, but it can have a higher cost because of the solvent recovery in this process. Some processes that may be used to separate the biomass to obtain lignin 16 can include any of the methods described in U.S. utility applications: A METHOD FOR PRODUCING BIOBASED CHEMICALS FROM PLANT BIOMASS (U.S. application Ser. No. 13/292,222 filed Nov. 9, 2011), A METHOD FOR PRODUCING BIOBASED CHEMICALS FROM WOODY BIOMASS (U.S. application Ser. No. 13/292,437 filed Nov. 9, 2011), A METHOD FOR PRODUCING BIOBASED CHEMICALS FROM AGRICULTURAL BIOMASS (U.S. application Ser. No. 13/292,531 filed Nov. 9, 2011), and A METHOD FOR PRODUCING BIOBASED CHEMICALS FROM CULTIVATED PLANT BIOMASS (U.S. application Ser. No. 13/292,632 filed Nov. 9, 2011). Another process to obtain lignin 16 that may be used at cellulosic ethanol refineries 10 may include acidic hydrolysis and/or enzymatic reactions. Typically, the lignin 16 recovered from the cellulosic ethanol refineries 10 may be used as boiler fuel. Additionally, the lignin 16 recovered from the cellulosic ethanol refineries 10 may undergo a pretreatment prior to entry into the process(es) described herein. The purpose of this lignin pretreatment may be to remove unwanted impurities from the lignin 16 and may include a series of steps to further separate lignin 16 from the other components of biomass such as cellulose and hemicellulose as well as the fats, oils, resins, pitches, waxes, other extractables that may be present in the biomass, or the salts, enzymes, and cellular debris that may contaminate the lignin from biomass processing. A lignin pretreatment process is described in detail in A METHOD FOR PRODUCING BIOBASED CHEMICALS FROM PLANT BIOMASS (U.S. application Ser. No. 13/292,222 filed Nov. 9, 2011).

Besides the other sources for lignin 16, sugar cane mills 12 may also provide lignin 16 used in the process described herein. sugar cane mills 12 can include bagasse by-product from sugar cane processing to produce sugar. Bagasse, the fibrous matter that remains after sugarcane or sorghum stalks are crushed to extract their juice, may often be used as a primary fuel source for sugar mills. The bagasse may be burned, producing sufficient heat energy to supply all the needs of a typical sugar cane mill. However, there may be an excess of bagasse when the energy supply to the sugar can mill has been provided.

Yet another source of lignin 16 may be lignin residue biomass 14. Lignin residue biomass 14 can include the lignin residue caustic solution by-product or recovered solid depolymerized lignin residue from tiered biobased chemical and biofuel production described in A METHOD FOR PRODUCING BIOBASED CHEMICALS FROM PLANT LIGNIN (U.S. application Ser. No. 13/453,422 filed Apr. 23, 2012).

Although several sources for lignin are presented herein, those sources for lignin are not limited to those listed. Any lignin 16 provided may be used within the process described to create value-added product(s). Producing these chemicals may provide a reduction in the costs associated with waste disposal of lignin 16 and a means to generate income from biobased chemical production. Besides waste product sources of lignin 16 for the recovered biomass 4, lignin 16 waste from the lignin 16 processing may also provide a source for producing energy. This waste may include recovered plant biomass waste lignin, kraft pulp mill waste lignin, sulfite pulp mill waste lignin, soda pulp mill waste lignin, cellulosic ethanol refinery waste lignin, sugar cane mill waste lignin, and commercial biomass fractionators waste lignin. In this reduction of waste for the process described herein, the waste product of the lignin biomass may be less than 30% of the lignin weight. It may also be less than 20% of the lignin weight. It may also be less than 10% of the lignin weight. These waste products, although reduced, may be used to produce energy which utilizes the waste product, providing value to the process. This energy production may be heat and/or power.

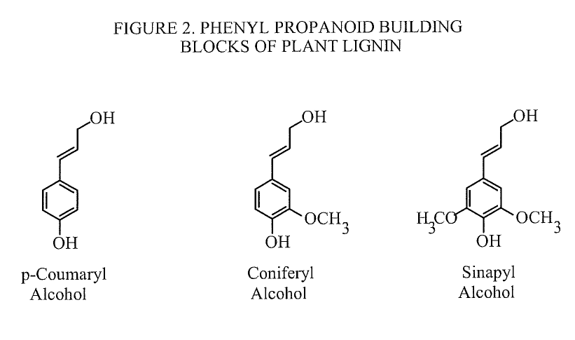

FIG. 2 provides some of the chemical building blocks of lignin 16. Lignin 16 constitutes one of the three major components of lignocellulosic biomass, of which the other two major components are cellulose and hemicellulose. The polymeric structure of lignin 16 can be very complex and a complete structure elucidation of any single lignin is still unknown. The building block compositions of lignin, the extent of polymerization, and the abundance of lignin alter from plant species to plant species. This composition may provide control of the composition of the biobased product(s) from lignin 16. The abundance of lignin in plants generally may decrease from softwoods to hardwoods, and also may decrease from hardwoods to grasses. Moreover, lignin structure can be impacted by the treatment process used to separate lignin from the other components of biomass.

Lignin 16 can be an amorphous polymer made up of three phenyl propanoid building blocks shown in FIG. 2. These building blocks may differ in the degree of oxygen substitution on the phenyl ring. In nature, lignin can impart strength and rigidity to the plant by extensive cross linking with polymeric hemicellulose and/or cellulose.

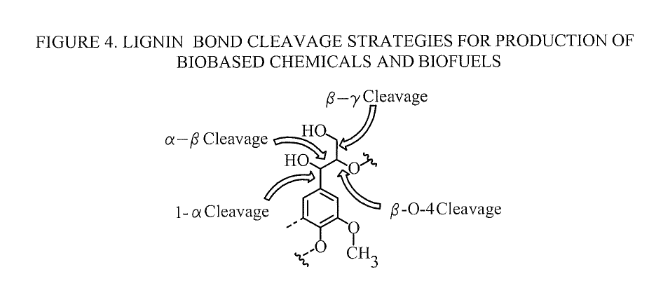

Most plant lignin 16 types may be comprised of all three building blocks shown in FIG. 2. Depending on the species of plant, the ratio of these three building blocks may vary. The composition of lignin may frequently be stated in terms of its 4-hydroxyphenyl (H), guaiacyl (G), and sinapyl (S) content. These aromatic systems can correspond, respectively, to the p-coumaryl alcohol, coniferyl alcohol, and sinapyl alcohol building blocks of lignin. First, the p-coumaryl alcohol building block may correspond to the p-hydroxyphenyl (H) make-up of lignin. Grassy plants like wheat straw and corn stover may tend to have the highest H contents. Two H-derived oxidation products of lignin may include 4-hydroxybenzaldehyde and 4-hydroxybenzoic acid. Second, the coniferyl alcohol building block may correspond to the guaiacyl (G) make-up of lignin. Softwoods like spruce and pine, in general, may tend to have the highest G content, often in excess of about 80% of the plant lignin. Two G-derived oxidation products of lignin may include vanillin and vanillic acid. Third, the sinapyl alcohol building block may correspond to the sinapyl (S) make-up of lignin. Typically, hardwoods have high S contents, which can often be over 50% where the balance may be comprised predominantly of G. Some examples of hardwoods may include willow and oak. Two S-derived oxidation products of lignin may include syringaldehyde and syringic acid. The values provided below in Table A may provide normalized H:G:S ratios found in certain lignin 16 by selective α-β cleavage lignin oxidative depolymerisation cleavage of the C9 phenyl propanoid building blocks (the α-β cleavage is described further in FIG. 4):

TABLE A
H:G:S Normalized Percentage Ratios of Various Plant Lignins

| | | | | |
|---|---|---|---|---|
| Entry | Plant Group/Species | % H | % G | % S |

Hardwoods

| | | | | |
|---|---|---|---|---|
| 1 | Eucalyptus grandis | 2 | 36 | 62 |
| 2 | Red Oak | ND | 37 | 63 |
| 3 | Cottonwood | ND | 45 | 55 |
| 4 | Sweet Gum | ND | 41 | 59 |
| 5 | Acacia | ND | 49 | 51 |
| 6 | Birch | ND | 28 | 72 |
| 7 | Red Alder | ND | 45 | 55 |
| 8 | Maple | ND | 48 | 52 |
| 9 | Salix integra | ND | 34 | 66 |
| 10 | Poplar | ND | 37 | 63 |

Softwoods

| | | | | |
|---|---|---|---|---|
| 11 | Softwood Kraft | 8 | 85 | 7 |
| 12 | Black Spruce | 7 | 84 | 7 |

Agricultural

| | | | | |
|---|---|---|---|---|
| 13 | Wheat Straw, milled only | 53 | 40 | 7 |
| 14 | Wheat Straw, alkaline treatment | 27 | 63 | 10 |
| 15 | Wheat Straw, acid treatment | 45 | 46 | 9 |
| 16 | Rice Straw | 33 | 46 | 21 |
| 17 | Corn Stover | 40 | 31 | 7 |

* ND = Not detected and/or not reported

Based on the lignin provided, the product distribution may parallel the H:G:S ratio. Selection of the lignin source may therefore allow for the prediction of a certain product ratio. For example, if high levels of G-derived products are desired, then a lignin composition of high G content may be preferred. These high-level of G-derived products may be obtained from either a specific lignin, which may include a specific plant species or biomass pretreatment method, that may provide a lignin of high G content and/or a blend of different lignin forms such that the blend has the desired G content.

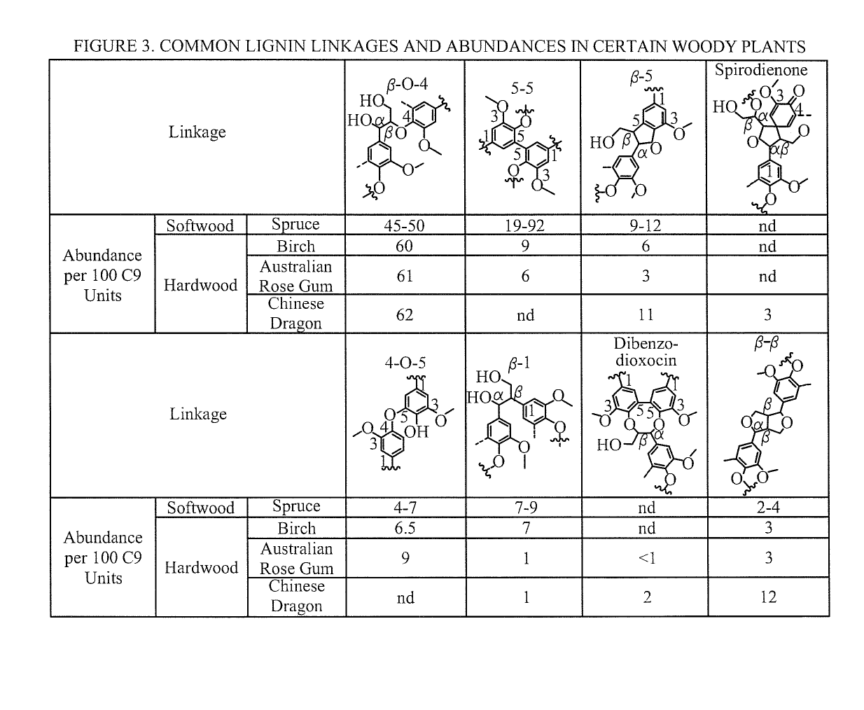

Besides the different H:G:S ratios from the different species, there may also be a difference in the H:G:S ratio after the biomass pretreatment method, even within the same plant species (see Table A wheat straw entries 13-15). For these different plant species and also lignin obtained from different biomass pretreatment methods, many different chemical linkages may occur between the three building blocks. Some of these common linkages may be seen in FIG. 3.

No matter the lignin 16 source or type of treatment, the polymeric structure of lignin may be complex and a complete structure for any single lignin is unknown. Further, samples of lignin obtained from a single lignin source may also differ in its polymeric structure, providing variable building block compositions even within the same sample.

FIG. 3 provides some common linkages and abundances in certain woody softwood and hardwood plants. The chart provides estimates as to the abundance of a particular linkage within some specific species. Although numbers have been provided, these numbers may vary due to other factors which may include but are not limited to lignin treatment, growth rate of the plant, region where growth of the plant occurs, and/or genetic differences of the plant.

For the lignin linkages of these softwoods and hardwoods, there may be at least 8 different linkages which may be commonly found. These linkages may include: β-O-4, 5-5, β-5, 4-O-5, β-1, β-β, spirodienone, and dibenzodioxocin. The abundance of these linkages may be measured by their prevalence per 100 C9 units.

The predominant linkage structure in lignin may be a β-O-4 linkage. This linkage may account for about 45% to about 60% or more of all linkages in woody lignin. In other types of plant lignin, this number may vary. For example, this linkage may reach about 80% or more in corn stover lignin. The linkage designation of β-O-4 can refer to a carbon-oxygen bond between the β-carbon, which is the central carbon of the propyl side chain of one building block, with the 4-hydroxy group on the phenyl ring of a second lignin phenyl propanoid building block. A β-O-4 linkage may occur between and among H, G, and S building blocks.

Another notable linkage may be the 5-5 linkage. The 5-5 linkage type can refer to a carbon-carbon bond between C-5 positions of two phenyl rings of two different phenyl propanoid building blocks. This linkage type may be common in some softwoods, but may not be as prevalent in some hardwoods. A 5-5 linkage may occur between and among H and G building blocks. A S building block may not enter into a 5-5 linkage because the C-5 position of S is occupied by a methoxy group and prevents this linkage.

The β-5 linkage may also be found in both softwoods and hardwoods. The β-5 linkage can refer to a carbon-carbon bond between the β-carbon position of one building block and C-5 position of the phenyl ring of a second building block. A β-5 linkage may occur between and among H, G, and S building blocks, although the building block comprising the C-5 linkage position may not be S because the C-5 position of S is occupied by a methoxy group and prevents this linkage.

The 4-O-5 linkage may be another linkage found in certain woody plants. The 4-O-5 linkage can refer to an ether linkage, which can comprise an oxygen-carbon bond, between a 4-hydroxyphenyl group of one building block with the C-5 position on a phenyl ring of a second building block. A 4-O-5 linkage may occur between and among H, G, and S building blocks, although the building block comprising the C-5 linkage position may not be S because the C-5 position of S is occupied by a methoxy group and prevents this linkage.

Another linkage may also include a β-1 linkage. The β-1 linkage can occur through a carbon-carbon bond between the β-carbon of one building block and position 1 of another phenyl ring. A β-1 linkage may occur between and among H, G, and S building blocks.

Yet another linkage may include a β-β linkage. A β-β linkage can be a carbon-carbon bond between the β positions of two building blocks, generally leading to a fused bis-furan system. This linkage may occur between and among H, G, and S building blocks

Some other linkages, although not as common or prevalent as some of the aforementioned linkages, may be the spirodienone and dibenzodioxocin linkages. The spirodienone and dibenzodioxocin linkages can be multifunctional linkages. These linkage types, however, may not be seen across all lignin types. The spirodienone linkage may occur between and among any of the three building blocks, shereas the biobenzodioxocin linkage may only occur with H or G because the C-5 position of S is occupied by a methoxy group and prevents this linkage.

To note, those linkages in FIG. 3 may be commonly found linkages in certain woody plants. They are, however, not an exhaustive list of all linkages found. Further, not all linkages can be seen in every lignin type, and the ratio of these linkages may change between different plant species and between different lignin pretreatments even within the same plant species.

FIG. 4 depicts some carbon-carbon and carbon-oxygen bond cleavage strategies for the production of biobased chemicals and/or biofuelsfor the most structurally common β-O-4 linkage. Different types of lignin 16 may also have different linkages between the building blocks to make-up the polymeric structure. Determining the quantity of certain linkage types from the lignin 16 source may selectively allow for the production of certain end-products. Especially in the design of an efficient biobased chemical production process, the cleavage of the structural linkages of lignin 16 may be selected such that specific products may be produced.

The most common phenyl propanoid linkage type for lignin 16 may be β-O-4, which can account for typically about 50% or more of all linkages in lignin 16. The β-O-4 linkage can refer to a bond between the β carbon, which can be the central carbon of the propyl side chain, and the 4-hydroxy group on the aryl ring of a second lignin building block. Other linkages may frequently occur in lignin 16, including 5-5, β-5, 4-O-5, β-1, β-β, spirodienone and dibenzodioxocin. However, not all linkages may be seen in every lignin 16 type, and the ratio of these linkages can change between the different lignins 16. The four potential carbon-carbon bond cleavages of a C9 phenyl propanoid backbone shown in FIG. 4 are:
1. No carbon-carbon cleavage, which may leave a C9 fragment (phenyl ring with a C3 side chain).
2. The β-γ cleavage, which can yield a C8 fragment (phenyl ring with a C2 side chain) and a C1 alkyl fragment.
3. The α-β cleavage, which may yield a C7 fragment (phenyl ring with a C1 side chain) and a C1 and/or C2 alkyl fragment.
4. The 1-α cleavage, which can yield a C6 fragment (phenyl ring) and a C1, C2, and/or C3 alkyl fragment.

A few specific examples of biobased chemicals listed in A METHOD FOR PRODUCING BIOBASED CHEMICALS FROM PLANT LIGNIN (U.S. application Ser. No. 13/453,422 filed Apr. 23, 2012) may include, but are not limited to, certain chemicals. In particular, the type of each carbon-carbon cleavage may result in a specified chemical being produced. For no carbon-carbon cleavage, the resulting chemicals may include, but are not limited to, propylbenzene, 1-phenyl-1-propene, 1-phenyl-2-propene, propylcyclohexane, propylcyclohexene, eugenol, isoeugenol, syringeugenol, iso-syringeugonol, propylcyclohexane, 4-propylphenol, 2-methoxy-4-propylphenol, 2,6-dimethoxy-4-hydroxyphenol, 3-(4-hydroxyphenyl)propionic acid, 3-(4-hydroxy-3-methoxyphenyl)propionic acid, 3-(4-hydroxy-3,5-dimethoxyphenyl)propionic acid, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnamic acid, 4-hydroxy-3-methoxycinnamic acid, and/or 4-hydroxy-3,5-dimethoxycinnamic acid. For a β-γ carbon-carbon cleavage, the chemicals formed may include, but are not limited to, ethylbenzene, styrene, ethylcyclohexane, ethylcyclohexene, 4-hydroxystyrene, 3-methoxy-4-hydroxystyrene, 3,5-dimethoxy-4-hydroxystyrene, 4-ethylphenol, 2-methoxy-4-ethylphenol, 2,6-dimethoxy-4-ethylphenol, 1-(4-hydroxyphenyl)ethanone, 1-(4-hydroxy-3-methoxyphenyl)ethanone, 1-(4-hydroxy-3,5-dimethoxy)ethanone, (4-hydroxyphenyl)acetaldehyde, (4-hydroxy-3-methoxyphenyl)acetaldehyde, (4-hydroxy-3,5-dimethoxyphenyl)acetaldehyde, (4-hydroxyphenyl)acetic acid, homovanillic acid, homosyringic acid, and/or formic acid. For an α-β carbon-carboncleavage, the chemicals formed may include, but are not limited to, toluene, methylcyclohexane, methylcyclohexene, 4-methylphenol, 2-methoxy-4-methylphenol, 2,6-dimethoxy-4-methylphenol, 4-hydroxybenzaldehyde, vanillin, syringaldehyde, 4-hydroxybenzoic acid, vanillic acid, syringic acid, acetic acid, glycolic acid, glyoxylic acid oxalic acid, and/or formic acid. With an 1-α carbon-carbon cleavage, benzene, phenol, guaiacol, 2,6-dimethoxyphenol, cyclohexane, cyclohexene, propanoic acid, lactic acid, malonic acid, acetic acid, glycolic acid, glyoxylic acid oxalic acid, and/or formic acid may result.

Likewise, multiple carbon-oxygen bond cleavage strategies may also exist since oxygen atom may be present at the α, and/or β, and/or γ carbons of the propyl side chain, as well as at the 3, and/or 4, and/or 5 positions of the phenyl ring. For instance, FIG. 4 depicts a β-O-4 cleavage since the β-O-4 linkage may be the most prevalent linkage structural connection in lignin. A β-O-4 cleavage may yield two C9 phenyl propanoids, each with the phenyl ring and a C3 side chain. Additionally carbon-oxygen bond cleavage may occur at the α and γ position of the side chain and at oxygen positions on the phenyl ring.

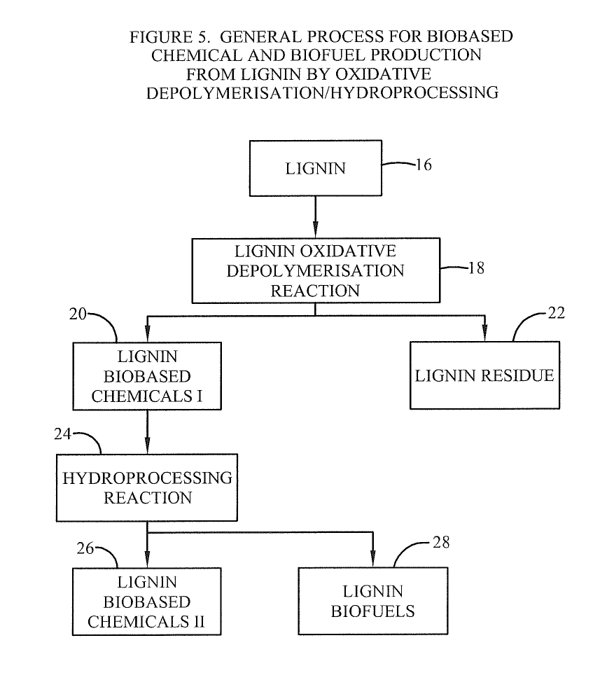

FIG. 5 provides a method for biobased chemical and/or biofuel production from lignin 16. The products from the lignin biomass may comprise at least one product of biobased chemicals, biofuels, and lignin residues. At least one of said products from said lignin biomass may also comprise at least two products of biobased chemicals, biofuels, and lignin residues. The biobased chemicals may comprise at least one chemical of commodity chemicals, fine chemicals, and specialty chemicals. Additionally, the biobased chemicals comprise at least one chemical of achiral chemicals, racemic chemicals, and chiral chemicals. This method may be provided from a type of process, namely chemical-induced processing, catalytic oxidative lignin depolymerisation processing, and catalytic hydroprocessing. These processes may be conducted in any order. Moreover, chemical-induced processing may take place in the same process as catalytic oxidative lignin depolymerisation processing and/or catalytic hydroprocessing. These processes may occur at a reaction temperature of about 50° C. to about 500° C., or may be performed at a reaction temperature of about 100° C. to about 250° C. Further, these processes may be induced by caustic, including at least one caustic of lithium hydroxide, sodium hydroxide, potassium hydroxide, cesium hydroxide, magnesium hydroxide, barium hydroxide, and calcium hydroxide. This caustic may also be carbonates and/or oxides of Group I and Group II metals of the Periodic Table. In FIG. 5, lignin 16 may be first subjected to a lignin oxidative depolymerisation reaction 18. This lignin oxidative depolymerisation reaction 18 step may break the lignin 16 polymer down into smaller fragments and specific biobased chemicals.

Several different methods may be used in the depolymerisation of lignin 16. These options may include gasification, pyrolysis, hydrogenolysis, oxidative depolymerisation, and/or hydrolysis. Although the methods provided above may allow for depolymerisation of lignin 16, some of these methods may provide more selective cracking of the bonds within the lignin 16, allowing for particular chemicals to be produced. Some of these methods may also be more efficient than others. The method may be chosen such that certain biobased chemical(s) may be produced.

The gasification of lignin may be one method for depolymerizing lignin. Gasification may degrade the carbocyclic backbone of lignin into low molecular weight gaseous products such as hydrogen, carbon monoxide, carbon dioxide and/or methane. In order to produce biobased chemicals in this manner, these gases may have to be subsequently converted back into aromatic and/or aliphatic compounds by multistep, complex secondary processes. Although gasification can be used in the production of biobased chemicals, the complex nature of this process may reduce its efficiency when compared to other potential processes.

Yet another method for lignin depolymerisation can be pyrolysis. Pyrolysis may convert lignin into gases, liquid oil (also known as bio-oil), and/or tar and char. One type of pyrolysis, thermolysis, may be a pyrolytic procedure performed at temperatures of about 200° C. to about 900° C. and in the absence of air so the lignin structure may be fragmented into smaller molecular weight units without significant combustion into carbon dioxide. The product distribution of thermolysis can be influenced by lignin feedstock type, the heating rate, the final depolymerisation temperature, and/or additives. The primary gaseous products of lignin thermolysis may be carbon monoxide, carbon dioxide, and/or methane. The liquid oil fraction may consist of methanol, acetone, acetaldehyde, mono-lignols, and/or mono-phenols and poly-substituted phenols. While the complex composition of the bio-oil can present the potential for production of chemicals from lignin, the economic separation of pure compounds from this mixture may be a significant economic challenge. Although thermolysis may provide a considerable amount of water from the dehydration of lignin, many of the volatile products may be water soluble, which can involve an additional step to remove the volatile product(s) from the waste water to prevent environmental pollution. Additionally, certain amounts of tar and char in the reactor may be formed in the reactor, requiring yet another step involving a cleaning of the reactor.

A third method for depolymerizing lignin may be hydrogenolysis, which occurs when pyrolysis occurs in the presence of hydrogen and/or a hydrogen-donating liquid. Catalysts and solvents may be employed to speed up the depoymerization reaction and may increase the yield of bio-oil. Solvents may include water, ethanol, propanol, isopropanol, acetonitrile, and/or ionic liquids. Typical reaction temperatures for hydrogenolysis can be about 300° C. to about 600° C., which may be lower than the temperatures used in lignin thermolysis. Lignin hydrogenolysis tends to afford a higher net conversion, a higher yield of mono-phenols, and less char formation relative to thermolysis. Depending upon the catalyst and hydrogen source, the obtained bio-oil consists of a mixture of monomeric, dimeric and oligomeric phenolic products. Using hydrogenolysis may provide considerable amounts of the dimeric and/or oligomeric phenolic products, potentially reducing the yield of biobased chemical products.

Oxidative depolymerisation can also be another method for the depolymerisation of lignin 16. The lignin oxidative depolymerisation reaction 18 may provide an efficient means for lignin 16 depolymerisation. This reaction step breaks the lignin 16 polymer down into smaller fragments and specific biobased chemicals. The lignin oxidative depolymerisation reaction 18 may allow for oxidative cracking of the lignin 16 by strong oxidants like hydrogen peroxide, which can lead to low molecular weight carboxylic acids such as, but not limited to, oxalic acid, formic acid, acetic acid, malonic acid, and/or succinic acid. Aromatic aldehydes and carboxylic acids may be intermediates in the oxidative degradation of lignin 16 with hydrogen peroxide; however, only trace amounts of these products may be analytically detected due to their rapid oxidative degradation. The lignin oxidative depolymerisation reaction 18 may also be completed using a catalyst, called catalytic oxidative lignin depolymerization processing. For example, the oxidation of lignin 16 with selective 1-α or α-β bond scission may be possible using a zirconium oxide-alumina-iron oxide catalyst in a fixed bed flow reactor, providing yields for benzene, toluene, xylene, cylcohexane, methylcyclohexane, 4-methylphenol, 2-methoxy-4-methylphenol, 2,6-dimethoxy-4-methylphenol, phenol, 2-methoxyphenol, and/or 2,6-dimethoxyphenol. Several other oxidation systems may also provide for selective oxidation of lignin 16. These other oxidation systems may include oxygen, nitrobenzene, permanganate, and/or soluble and immobilized transition metal catalyst systems. With the lignin oxidative depolymerisation reaction 18, several different processes in which the bond cleavage can be controlled may provide specific end products. For instance, a process targeting 1-α bond cleavage of the lignin backbone may deliver access to a biobased benzene, phenol, 2-methoxyphenol, and/or 2,6-dimethoxyphenol. In addition, a process targeting an oxidative α-β bond cleavage of the lignin skeleton may afford entry to a biobased toluene, xylene, methylcyclohexane, 4-methylphenol, 2-methoxy-4-methylphenol, and/or 2,6-dimethoxy-4-methylphenol. Overall, a lignin oxidative depolymerisation reaction 18, in principal, may provide for an efficient use of the lignin's carbon utilization. This lignin oxidative depolymerisation reaction 18 step may afford a lignin biobased chemicals I 20 product(s), and depending upon the extent of the lignin oxidative depolymerisation reaction 18, a certain amount of a lignin residue 22. The processing described in FIG. 5 can be a batch or flow operation.

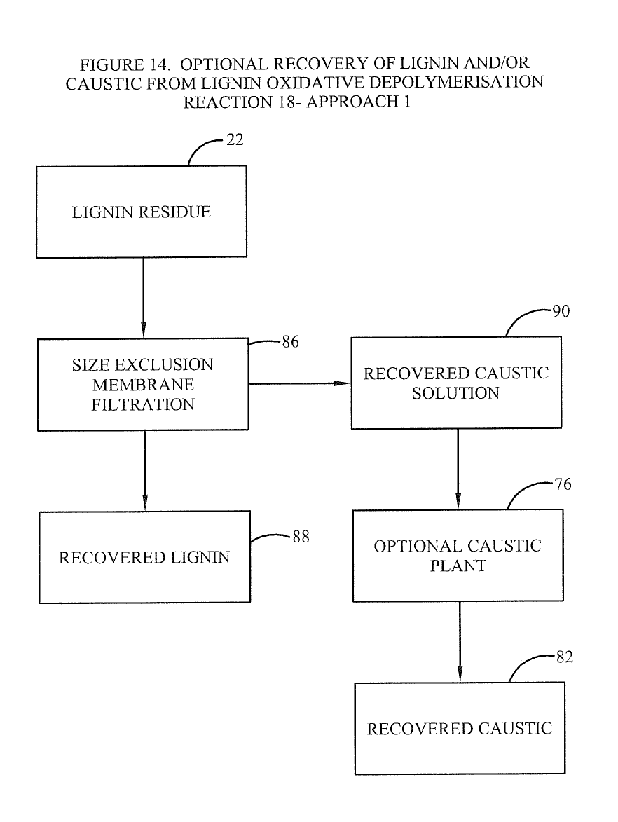

The amount of lignin residue 22 from the lignin oxidation depolymerisation reaction 18 can range from more than about 90% to less than about 10% of the original amount of lignin 16 entering the process. It may also range from about 50% to about 10% of the original amount of lignin 16 entering the process. Lignin residue 22 may be sent (a) for heat and power generation, and/or (b) for recycling as lignin residue biomass 14 for further use as lignin 16, and/or (c) transformed into tiered biobased chemicals and biofuels as described in A METHOD FOR PRODUCING BIOBASED CHEMICALS FROM PLANT LIGNIN (U.S. application Ser. No. 13/453,422 filed Apr. 23, 2012) as well as FIGS. 14 and/or 15.

If lignin biobased chemicals II 26 and/or lignin biofuels 28 are the desired products from lignin 16, then the lignin biobased chemicals I 20 may be subjected to a hydroprocessing reaction 24. The purpose of the hydroprocessing reaction 24 may be to hydrodeoxygenate the intermediary lignin biobased chemicals I 20. The hydroprocessing reaction 24 can be the catalytic transformation of oxygenated biomass into hydrocarbons and water, resulting in catalytic hydroprocessing. Depending upon the lignin oxidative depolymerisation reaction 18, different hydroprocessing reaction 24 catalysts and processing conditions may be used. The hydroprocessing reaction 24 may be conducted in the presence of high-pressure hydrogen, or hydrogen-donating liquids, at a temperature of about 200° C. to about 500° C. The hydrogen in this process may serve to reductively remove oxygen from the C—O bonds of biomass. Considerable efforts have turned to the development of selective hydrodeoxygenation catalysts since over-reduction of the substrate consumes valuable hydrogen. Several hydrodeoxygenation catalyst types may be chosen, but care must be given in the selection of such a catalyst since a catalyst may be deactivated or may produce an undesired chemical. Some of these catalysts may include alumina supported sulfided molybdenum catalyst, zirconia and sulfated zirconia supported noble metals and bimetallic catalysts, boron-promoted bimetallic catalysts, transition metal phosphides, transition metal carbides, and/or bifunctional zeolite supported noble metal catalysts. Further, the hydroprocessing reaction 24 step may use metal catalysis either with or without added caustic. The hydroprocessing reaction 24 can be a batch or flow operation.

FIG. 6 provides a detailed listing of various chemicals derived from the conversion of lignin 16 to lignin biobased chemicals I 20, lignin biobased chemicals II 26, and/or lignin biofuels 28.

The first group of chemicals derived from lignin 16 may be lignin biobased chemicals I 20. Lignin biobased chemicals I 20 can include chemicals produced by oxidation of lignin, and products derived therefrom. These aryl products may retain the oxygen functionality of aromatic portion of phenyl propanoid structure, as well some or most of the oxygen functionality of the side chain. The alkyl carboxylic acids may also retain some or all of the functionality of the propanoid chain.

For lignin biobased chemicals I 20, lignin 16 may provide at least one chemical of aryl aldehydes, aryl carboxylic acids, aryl ketones, and alkyl carboxylic acids. Further, specific chemicals may include 4-hydroxybenzaldehye, vanillin, syringaldehyde, 4-hydroxybenzoic acid, vanillic acid, syringic acid, (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, 4-hydroxy-3,5-dimethoxycinnaminaldehyde, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and/or 4-hydroxy-3,5-dimethoxycinnamic acid. Aryl esters comprising a C₁-C₁₆ ester may include at least one chemical of 4-hydroxybenzoic acid, vanillic acid, syringic acid, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and/or 4-hydroxy-3,5-dimethoxycinnamic acid. Some other tier 1 lignin biobased chemicals 16 may also include aryl ketones comprising at least one chemical of 1-(4-hydroxyphenyl)ethanone, 1-(4-hydroxy-3-methoxyphenyl)ethanone, 1-(4-hydroxy-3,5-dimethoxyphenyl)ethanone, 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, and/or 1-(4-hydroxy-3,5-dimethoxyphenyl)-2-propanone. Moreover, some tier 1 lignin biobased chemicals 16 may include at least one chemical from aliphatic carboxylic acids comprised of formic acid, oxalic acid, acetic acid, glycolic acid, glyoxylic acid, propionic acid, lactic acid, and malonic acid.

The next group of chemicals derived from lignin 16 may be lignin biobased chemicals II 26. Lignin biobased chemicals II 26 can comprise six main groups of chemicals depending upon the degree of remaining oxygen functionality after hydroprocessing 24: phenols, aromatic hydrocarbons, cycloalkanes and cycloalkenes, alkanes and alkenes, alkyl carboxylic acids and alkyl esters, and performance chemicals. The first group can be the phenol group. For the phenol group, the phenols, alkyl phenols, and/or alkenyl phenols may retain some or all the aromatic ring oxygenation patterns of the phenyl propanoid building blocks of lignin. The second group can be referred to as the aromatic hydrocarbon group. These aromatic hydrocarbons may comprise benzene, toluene, xylenes, mestitylenes, biaryls, aryl alkanes and/or aryl alkenes, which may be formed upon losing the aromatic ring oxygenation patterns of the phenols, alkyl phenols, and/or alkenyl phenols. The third group can comprise the cycloalkanes and/or the cycloalkenes. For the third group, the cycloalkanes and/or cycloalkenes may be further reduction products of the benzene, toluene, xylenes, mesitylenes, biaryls, aryl alkanes and aryl alkenes. The fourth group can comprise alkanes and alkenes. The alkanes and alkenes may be formed by deoxygenation of the lignin propanoid side chain and fragmentative ring openings. The fifth group can comprise the alkyl carboxylic acids and alkyl esters. The alkyl carboxylic acids and alkyl esters may be formed by oxidation of the propanoid side chain and/or alkanes and alkenes. The last group is the performance chemicals. The performance chemicals may comprise mixtures or defined blends of phenols, aromatic hydrocarbons, cycloalkanes and cycloalkenes, alkanes and alkenes, and/or alkyl esters

For lignin biofuels 28, lignin 16 may provide similar chemicals to lignin biobased chemicals II 26 in that there may be at least one chemical of phenols, alkyl phenols, alkenyl phenols, benzene, toluene, xylenes, mesitylenes, biaryls, aryl alkanes, aryl alkenes, alkanes, alkenes, cycloalkanes, cycloalkenes, alkyl esters, and performance chemicals. Lignin biobased chemicals II 26 may also comprise product mixtures of biobased chemicals of similar boiling point range. Additionally, lignin biofuels 28 may also comprise some blends of phenols, alkyl phenols, alkenyl phenols, benzene, toluene, xylenes, mesitylenes, biaryls, aryl alkanes, aryl alkenes, alkanes, alkenes, cycloalkanes, cycloalkenes, and/oralkyl esters. Further, the blends of the lignin biofuels 28 may comprise product mixtures of biobased chemicals with a carbon and hydrogen content of about 80% to about 100%. Also, blends of the lignin biofuels 28 may comprise product mixtures of biobased chemicals with research octane number of at least about 90.

For lignin biobased chemicals II 26 and/or lignin biofuels 28, specific chemicals may include phenol, guaiacol, 2,6-dimethoxyphenol, 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, 2,4,6-trimethylphenol, 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, 2,6-dimethoxy-4-t-butylphenol, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and/or iso-syringeugenol.

The lignin biobased chemicals II 26 and/or lignin biofuels 28 may also include alkyl phenols comprising at least one of general molecular structure:

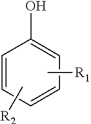

wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₁ and R₂ are located at positions 2, 3, 4, or 5 of the phenol ring.

The lignin biobased chemicals II 26 and/or lignin biofuels 28 may also include alkyl phenols comprising at least one of general molecular structure:

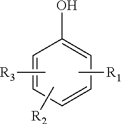

wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

For the lignin biobased chemicals II 26 and/or lignin biofuels 28, specific chemicals may include benzene, toluene, 1,2-dimethylbenzene, 1,3-dimethylbenzene, 1,4-dimethylbenzene, 1,2,3-trimethylbenzene, 1,2,4-trimethylbenzene, 1,3,5-trimethylbenzene, ethylbenzene, propylbenzene, isopropylbenzene, butylbenzene, isobutylbenzene, t-butylbenzene, styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, 1-(4-methylphenyl)-2-propene, hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, 3,4,5-trimethylnonane, cyclopentane, cyclohexane, cycloheptane, methylcyclopentane, methylcyclohexane, methylcycloheptane, ethylcyclopentane, ethylcyclohexane, ethylcycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and/or 1,4-dimethylcycloheptane.

For the aryl alkanes derived from lignin biobased chemicals II 26 and/or lignin biofuels 28, at least one chemical of a general molecular structure may comprise:

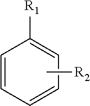

wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₂ is located at positions 2, 3, 4, or 5 of the ring.

For the aryl alkanes derived from lignin biobased chemicals II 26 and/or lignin biofuels 28, at least one chemical of a general molecular structure may comprise:

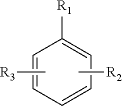

wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and butyl; and
wherein R₂ and R₃ are located at positions 2, 3, 4, or 5 of the ring.

For the biaryls derived from lignin biobased chemicals II 26 and/or lignin biofuels 28, specific chemicals may include biphenyl, 4,4′-dimethylbiphenyl, 3,3′-dimethylbiphenyl, 2,2′-dimethylbiphenyl, 3,4′-dimethylbiphenyl, 2,4′-dimethylbiphenyl, 2,3′-dimethylbiphenyl, 4,4′-diethylbiphenyl, 3,3′-diethylbiphenyl, 2,2′-diethylbiphenyl, 3,4′-diethylbiphenyl, 2,4′-diethylbiphenyl, 2,3′-diethylbiphenyl, 4,4′-dipropylbiphenyl, 3,3′-dipropylbiphenyl, 2,2′-dipropylbiphenyl, 3,4′-dipropylbiphenyl, 2,4′-dipropylbiphenyl, and 2,3′-dipropylbiphenyl.

For the cycloalkanes derived from lignin biobased chemicals II 26 and/or lignin biofuels 28, at least one chemical of a general molecular structure may comprise:

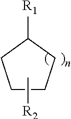

wherein n is 1, 2, or 3;
wherein R₁ is selected from among methyl, ethyl, propyl, isopropyl, butyl, isobutyl, and t-butyl;
wherein R₂ is selected from among ethyl, propyl, propyl, isopropyl, butyl, isobutyl, and t-butyl; and
wherein R₂ is located at any ring position other than that of R₁.

For the lignin biobased chemicals II 26 and/or lignin biofuels 28, lignin 16 may also provide at least one chemical of alkenes comprising at least one partially unsaturated alkane. Lignin 16 may also provide at least one chemical of cycloalkenes comprising at least one partially unsaturated cycloalkane. Additionally, performance chemicals may comprise at least one chemical of phenols, alkyl phenols, alkenyl phenols, benzene, toluene, xylenes, mesitylenes, biaryls, aryl alkanes, aryl alkenes, alkanes, alkenes, cycloalkanes, cycloalkenes, and alkyl esters.

For the alkyl esters derived from lignin biobased chemicals II 26 and/or lignin biofuels 28, at least on chemical of a general molecular structure may comprise:

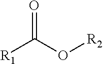

wherein R₁ and R₂ are selected from among methyl, ethyl, propyl, isopropyl, butyl, isobutyl, and t-butyl.

The lignin biofuels 28 described in FIG. 5 may also be blends of generally lignin biobased chemicals II 26 having 1) similar boiling point ranges, 2) similar carbon and hydrogen ratios, and/or 3) similar research octane numbers of at least about 90 that are useful for transportation and heating fuels, as described in A METHOD FOR PRODUCING BIOBASED CHEMICALS FROM PLANT LIGNIN (U.S. application Ser. No. 13/453,422 filed Apr. 23, 2012). For the lignin biofuels 22, the blends of lignin biofuels 22 can be provided for transportation fuels, heating fuels, and/or fuel additives. The transportation fuels can serve at least one market of automobile fuels, truck fuels, ship fuels, and aircraft fuels. The heating fuels may serve at least one market of home heating fuels, commercial heating fuels, and industrial boiler fuels. The fuel additives can serve at least one market of transportation fuels and heating fuels.

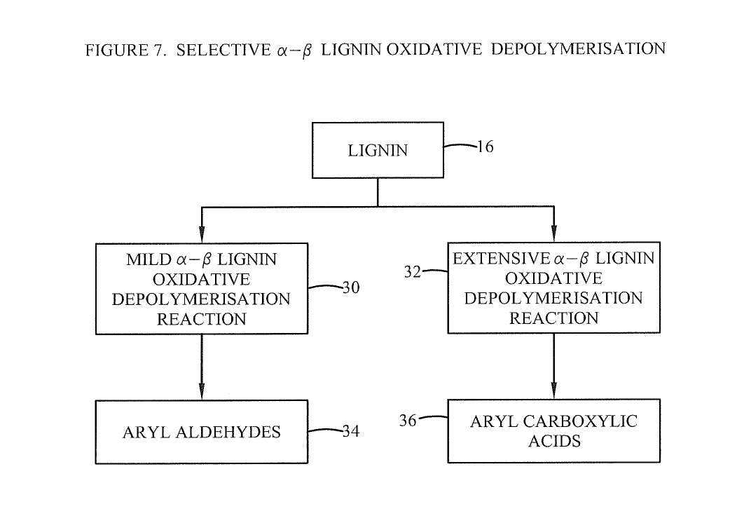

FIG. 7 provides at least two different options that may be available for the oxidative depolymerisation of lignin 16. These options may include a mild α-β lignin oxidative depolymerisation reaction 30 and/or an extensive α-β lignin oxidative depolymerisation reaction 32. The difference between these two options may be in the extent of oxidation. Using the mild α-β lignin oxidative depolymerisation reaction 30 and/or an extensive α-β lignin oxidative depolymerisation reaction 32 may provide methods in controlling the selection of the product(s) from the lignin 16. These α-β lignin oxidative depolymerisation reactions may either be selective or non-selective in providing products from lignin 16.

Selective α-β lignin oxidative depolymerisation, which may yield a C7 fragment and a C1 or C2 alkyl fragment, may provide entry to specific lignin biobased chemicals I 20 comprised of at least one chemical of aryl aldehydes 34 and aryl carboxylic acids 36. These specific lignin biobased chemicals I 20 may retain about 7 out of 9 carbons of the phenyl propanoid building block of lignin, which can equate to a natural carbon utilization of about 78% in the lignin biobased chemicals I 20 product.

These oxidative lignin depolymerisation options, either mild or extensive, can utilize metal catalysis either with or without added caustic. This caustic may be lithium hydroxide, sodium hydroxide, potassium hydroxide, cesium hydroxide, magnesium hydroxide, barium hydroxide, and/or calcium hydroxide. This caustic may also be carbonates and/or oxides of Group I and Group II metals of the Periodic Table. Both oxidative lignin depolymerisation processes, either mild or extensive, may utilize metal catalysts, either in the presence or absence of the caustic. The catalyst may be a homogeneous species, or a heterogeneous species, or a mixed metal system, or a metal system supported on an inert solid matrix. The metal catalyst may include, but is not limited to, various salts and complexes of the Periodic Table Group 3 through Group 12 transitions metals, and/or lanthanides, and/or actinides, as well as mixed metal systems thereof.

The oxidant for such reactions may be air, or oxygen, or hydrogen peroxide, or an organic peroxide, or an organic nitro compound, or mixtures thereof. The processing in either the mild la-β lignin oxidative depolymerisation reaction 30 and/or an extensive α-β lignin oxidative depolymerisation reaction 32 can be a batch or flow operation. The solvent system for this reaction may be aqueous, alcoholic, organic, ionic liquid based, or mixtures thereof. The reaction may be performed at a temperature from about 50° C. to about 300° C. The reaction may also be conducted at a temperature of about 100° C. to about 200° C.

Particularly under alkaline conditions, the β-O-4 cleavage of the lignin backbone may be prevalent along with the α-β lignin oxidative depolymerisation. The β-O-4 lignin cleavage may proceed prior to, in concert with, or subsequent to α-β-lignin depolymerisation. The β-O-4 cleavage may also be conducted as a separate processing step of the α-β-lignin oxidative depolymerisation. The β-O-4 cleavage may occur in the same reactor as α-β lignin oxidative depolymerisation, or it may be conducted in a separate reactor.

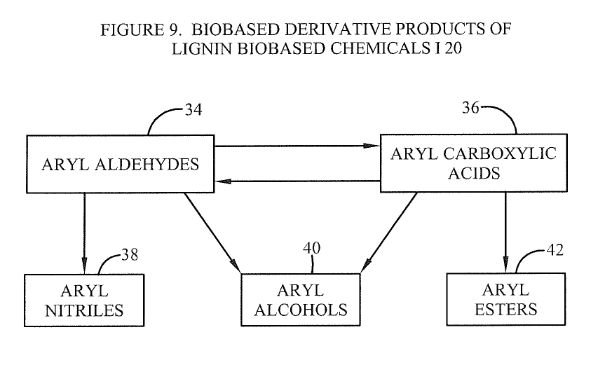

For the mild α-β lignin oxidative depolymerisation reaction 30, aryl aldehydes 34 may be the primary product group. Specific examples of aryl aldehydes 34 that can be produced by this process may include at least one chemical of 4-hydroxybenzaldehyde, vanillin, and syringaldehyde. These aryl aldehydes 34 may be formed by α-β lignin oxidative cleavage of the phenyl propanoids making up at least one of the β-O-4, and/or β-5, and/or β-1, and/or β-β, and/or spirodienone, and/or dibenzodioxocin linkages of lignin. The product yield from such a reaction may be from about 5% by weight to about 50% by weight or higher relative to the dried lignin weight. The aryl aldehyde 34 product mix from lignin oxidation may generally reflect the H:G:S ratio of the lignin 16, and can be controlled in part by the selection of lignin 16 feedstock (i.e., the plant species or biomass treatment method). Alternatively, a blend of lignin feedstock may be used to control the product distribution. Over-oxidation of the formed aryl aldehydes 34 product may lead to certain aryl carboxylic acids 36, and more specifically to at least one chemical of 4-hydroxybenzoic acid, vanillic acid, and syringic acid. In subsequent known chemistries that are described in FIG. 9, aryl aldehydes 34 may be converted to aryl carboxylic acid 36, and/or aryl nitriles 38, and/or aryl alcohols 40. As an example, vanillin can be converted to vanillic acid by oxidation, or into 4-hydroxy-3-methoxybenzyl alcohol by reduction. With mild α-β lignin oxidative depolymerisation reaction 30 and/or an extensive α-β lignin oxidative depolymerisation reaction 32, lignin 16 may be reacted to form aryl aldehydes 34 and aryl carboxylic acids 36.

For the extensive α-β lignin oxidative depolymerisation reaction 32, aryl carboxylic acids 36 may be the primary products. The product yield from such a reaction may be from about 25% by weight to about 100% by weight, relative to the dried lignin 16 weight. The product yield may also be from about 50% by weight to about 100% by weight, also relative to the weight of the dried lignin 16. The product mixture of the aryl carboxylic acids 36 from the extensive α-β lignin oxidative depolymerisation reaction 32 reflects the H:G:S ratio of the lignin 16, and may be controlled in part by the selection of lignin 16 feedstock. Alternatively, a blend of lignin feedstock can be used to control the product distribution. For example, the selection of the particular plant species or biomass pretreatment method may control the H:G:S ratio of the feedstock. Also in subsequent known chemistries that are described in FIG. 9, aryl carboxylic acids 36 can be converted into aryl aldehydes 34 and aryl alcohols 40 by reduction, and aryl esters 42 by esterification. In this manner, vanillic acid can be converted into vanillin or 4-hydroxy-3-methoxybenzaldehyde by reduction, or ethyl vanillate by esterification with ethanol.

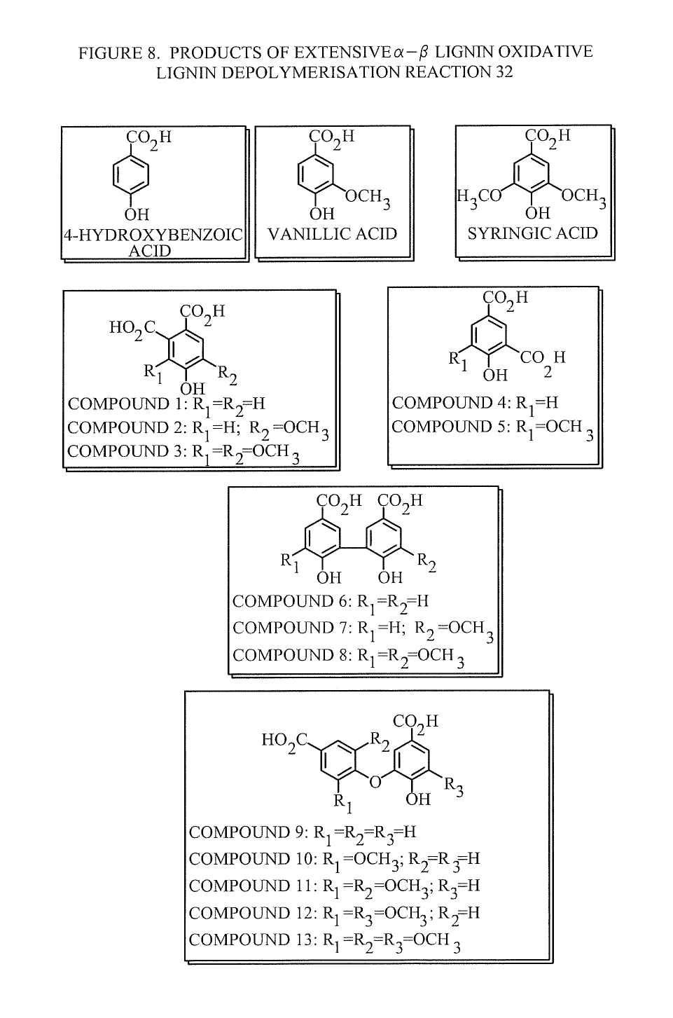

The major aryl carboxylic acids 36 products that may be formed by the extensive α-β lignin oxidative depolymerisation reaction 32 are shown in FIG. 8. Three specific examples of such products may be at least one aryl carboxylic acid 36 of 4-hydroxybenzoic acid, vanillic acid, and syringic acid. Selective α-β lignin oxidative depolymerisation has important commercial implications for selective transformations of lignin into large volume products that consist of at least one chemical of 4-methylphenols, toluene, xylene, and mesitylene. Such transformations may involve a subsequent hydroprocessing reaction 24 of the aryl aldehydes 34 and/or aryl carboxylic acids 36. The reason that selective α-β lignin oxidative depolymerisation can be so advantageous in the preparation of these high volume chemicals is that it can provide the C-7 structure of these products from lignin, while maximizing use of the carbon atoms provided by nature, and while minimizing products from other lignin cleavage modes that may complicate product isolation/purification.

Under oxidative depolymerisation, aryl carboxylic acids 36 could be formed through selective α-β bond cleavage of lignin and require hydrodeoxygenation of both the carboxylic acid function and the aromatic ring. Selective α-β lignin oxidative depolymerisation provides entry to Lignin biobased chemicals I 20 retaining 7 out of 9 carbons of the phenyl propanoid building block of lignin. This equates to a natural carbon utilization of 77% in the product. Under oxidative depolymerisation, the carbon utilization of lignin may also be at least 66%, 88%, or 100% depending upon the mode of cleavage in FIG. 4. This C-7 aromatic fragment of Selective α-β lignin oxidative depolymerisation has important implications for selective transformations into 4-methylphenols, toluene, and xylene from lignin 16 through a subsequent hydroprocessing reaction 24.

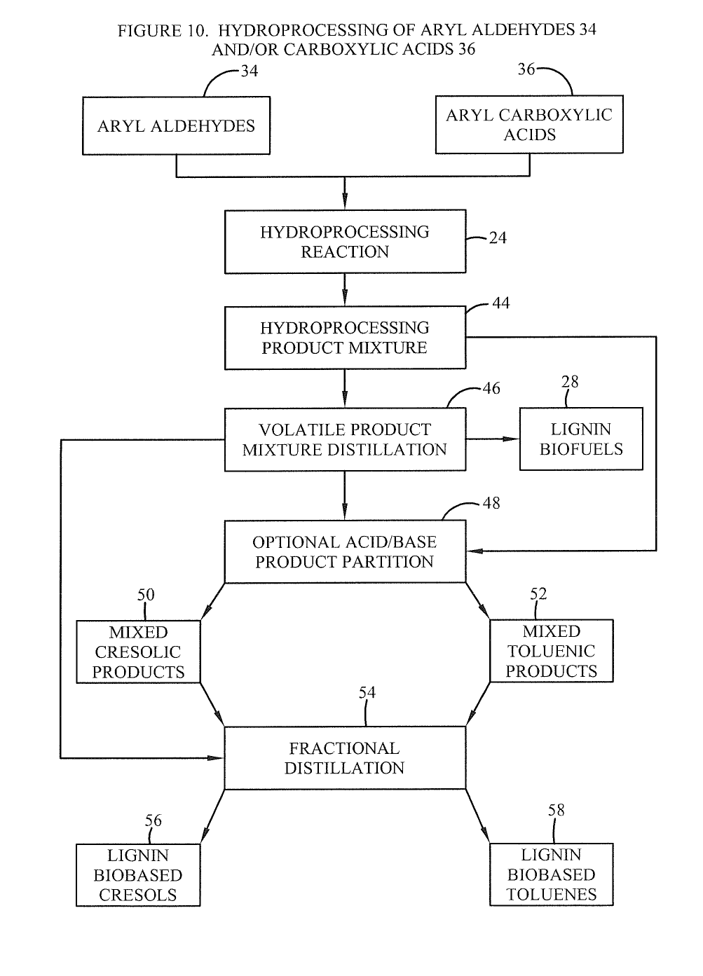

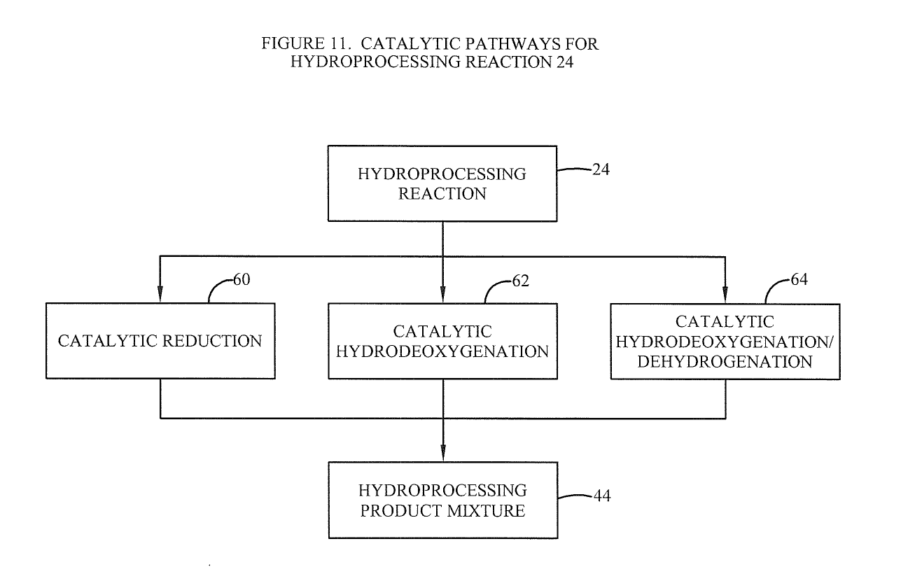

Hydrodeoxygenation can be the catalytic transformation of oxygenated biomass into hydrocarbons and water. These reactions may be conducted in the presence of high-pressure hydrogen, or hydrogen-donating liquids, at a temperature of about 200° C. to about 500° C. The role of hydrogen in this process can be to reductively remove oxygen from the C—O bonds of biomass. Several hydrodeoxygenation catalyst types are described below in FIGS. 10 and 11. In addition to the choice of catalyst, care may be given in selection of a support since phenolic compounds strongly chemisorb to certain supports such as alumina, leading to carbon deposition and catalyst deactivation. Depending upon the lignin depolymerisation process, different hydrodeoxygenation catalysts and processing conditions may be required. This is conceptually shown in FIG. 5 for a lignin oxidative depolymerisation reaction 18 and hydroprocessing reaction 24 approach to lignin biobased chemicals II and lignin biofuels 28. Under oxidative depolymerisation, aryl aldehydes 34 and/or aryl carboxylic acids 36 may be formed through selective α-β bond cleavage of lignin and require hydrodeoxygenation of both the carbonyl function and the aromatic ring. While not specifically depicted in FIG. 5, over reduction to cyclohexanols, cyclohexenes, and cyclohexanes may prove to be a more commercially viable hydrodeoxygenation process even though it would necessitate an additional dehydrogenation processing step to yield chemicals of the aromatic hydrocarbon group. Additionally, this reaction step can also utilize metal catalysis either with or without added caustic.

FIG. 8 provides some biobased chemicals that may be products of the extensive α-β lignin oxidative lignin depolymerization reaction 32. FIG. 7 depicts the extensive α-β lignin oxidative lignin depolymerization reaction 32 where aryl carboxylic acids 36 may be the primary products. In the extensive α-β lignin oxidative lignin depolymerization reaction 32, which may yield a C7 fragment (phenyl ring with a C1 side chain) and a C1 and/or C2 alkyl fragment, at least one chemical of 4-hydroxybenzoic acid, vanillic acid, and syringic acid may be formed by α-β oxidative cleavage of the phenyl propanoid making up the β-O-4, and/or β-5, and/or β-1, and/or β-β, and/or spirodienone, and/or dibenzodioxocin linkages of lignin.

At least one chemical of compounds 4 and 5 may be formed by an extensive α-β lignin oxidative depolymerisation reaction 32 of the phenyl propanoid making up a β-5 linkage. When an extensive α-β lignin oxidative depolymerisation reaction 32 of the dibenzodioxocin and 5-5 linkages can occur, chemical(s) of compounds 6, 7, and/or 8 may be formed. When an extensive α-β lignin oxidative depolymerisation reaction 32 of the 4-O-5 linkage may occur at least one chemical of compounds 9-13 as shown in FIG. 8 may arise.

Besides Compounds 4 and/or 5, the remainder of the compounds shown in FIG. 8 may be minor components of an extensive α-β lignin oxidative depolymerisation reaction 32.

Further, the abundances of the products shown in FIG. 8 may parallel the H:G:S ratio of the lignin 16 and can be influenced by the lignin 16 feedstock (i.e., the plant species) and/or lignin 16 pre-treatment method. Alternatively, the feedstock may be blended with different lignin 16 types to adjust the H:G:S ratio of the lignin 16 and achieve a desired product ratio.

FIG. 9 provides biobased derivative products of the lignin biobased chemicals I 20. The aryl aldehydes 34 and aryl carboxylic acid 36 products that may be produced by the biomass conversion methods claimed herein may be transformed into any number of value-added, biobased derivative products that are currently produced from petroleum. These derivatives products may comprise at least one of, but are not limited to, those chemical groups illustrated in FIG. 9.

For example, through application of known transformative chemistries, biobased aryl aldehydes 34 may be converted into biobased aryl carboxylic acid 36 by oxidation. Alternatively, aryl aldehydes 34 may be converted into biobased aryl nitriles 38 through the use of dehydration of an N-hydroxyimine intermediate. Moreover, a reduction of biobased aryl aldehydes 34 may provide biobased aryl alcohols 40. As an example, a biobased vanillin produced by lignin oxidation methods claimed herein may be converted into a biobased vanillic acid, and/or a biobased 4-hydroxy-3-methoxybenzonitrile, and/or a biobased 4-hydroxy-3-methoxybenzyl alcohol. The biobased derivative aryl aldehydes 34 may comprise at least one chemical of 4-hydroxybenzaldehyde, vanillin, syringaldehyde, and the bis-aldehyde derivatives, corresponding to compounds 1-13 of FIG. 8.

Also through application of known transformative chemistries, biobased aryl carboxylic acids 36 may be converted into aryl aldehydes 34 and aryl alcohols 40 by reduction chemistries and aryl esters 42 by esterification. In this manner, a biobased vanillic acid produced by the lignin oxidation methods claimed herein may also be converted into a biobased vanillin, and/or a biobased 4-hydroxy-3-methoxybenzyl alcohol, and/or a biobased ethyl vanillate. The biobased derivative aryl carboxylic acids 36 may comprise at least one chemical of 4-hydroxybenzoic acid, vanillic acid, and syringic acid and compounds 1-3 of FIG. 8

The biobased derivative aryl nitriles 38 may comprise at least one chemical of 4-hydroxybenzonitrile, 4-hydroxy-3-methoxybenzonitrile, 4-hydroxy-3,5-dimethoxybenzonitrile, and the bis-nitriles, also corresponding to compounds 1-13 of FIG. 8.

The biobased derivative aryl alcohols 40 may comprise at least one chemical of 4-hydroxybenzyl alcohol, 4-hydroxy-3-benzyl alcohol, 4-hydroxy-3,5-dimethoxybenzyl alcohol, and the bis-benzyl alcohols, also corresponding to compounds 1-13 of FIG. 8.

Finally, the biobased derivative aryl esters 42 may comprise a C1-C16 ester of at least one chemical of 4-hydroxybenzoic acid, vanillic acid, syringic acid, and compounds 1-13 of FIG. 8.

FIG. 10 details the hydroprocessing method. Using the aryl aldehydes 34 and/or aryl carboxylic acids 36 provided from the α-β lignin oxidative depolymerization methods from FIG. 7, the hydroprocessing reaction 24 may provide lignin biofuels 28, and/or lignin biobased cresols 56, and/or lignin biobased toluenes 58. In this instance, lignin biobased cresols 56 and/or lignin biobased toluenes 58 may correspond to examples of lignin biobased chemicals II 26 of FIG. 5. The hydroprocessing may be selective or non-selective in the reduction of lignin biomass and products of lignin biomass.

In the preparation of at least one chemical of lignin biobased cresols 56 and lignin biobased toluenes 58, either aryl carboxylic acids 36 and/or aryl aldehydes 34 may be treated by a hydroprocessing reaction 24. However, since the oxidative conversion of lignin into aryl carboxylic acids 36 may provide for a higher product yield than that for aryl aldehydes 34, the use of aryl carboxylic acids 36 in the hydroprocessing reaction 24 may be economically favored over the use of aryl aldehydes 34. However, the processing depicted in FIG. 10 may be a general process for either aryl carboxylic acids 36 or aryl aldehydes 34.

The hydroprocessing reaction 24 of aryl aldehydes 34 and/or aryl carboxylic acids 36 may provide a hydroprocessing product mixture 44. The hydroprocessing reaction 24 may be conducted with at least one chemical of aryl aldehydes 34 and/or aryl carboxylic acids 36, or with a mixture comprised of at least two chemicals of aryl aldehydes 34 and/or aryl carboxylic acids 36. These chemicals may be pure or impure substances.

For the hydroprocessing reaction 24, a catalytic reaction may be used. Depending on the preferred biobased end product(s), the type of catalytic reaction may differ. These catalysts may include a metal salt, a metal complex, and/or an elemental metal. These catalytic reactions within the hydroprocessing reaction 24, including catalytic reduction 60, catalytic hydrodeoxygenation 62, and/or catalytic hydrodeoxygenation/dehydrogenation 64, are detailed further in FIG. 11. Further, the hydroprocessing reaction may occur either in the presence or absence of added caustic. The caustic may include lithium hydroxide, sodium hydroxide, potassium hydroxide, cesium hydroxide, magnesium hydroxide, barium hydroxide, and/or calcium hydroxide. This caustic may also be carbonates and/or oxides of Group I and Group II metals of the Periodic Table. The hydroprocessing reaction 24 can be performed as either a batch or a flow process. Reaction temperatures for the hydroprocessing reaction 24 may be from about 50° C. to about 500° C. The reaction temperatures may also be conducted at a temperature of about 50° C. to about 300° C.

After the hydroprocessing reaction 24, a hydroprocessing product mixture 44 may be formed. The extent to which the hydroprocessing reaction 24 takes place may impact the product mixture obtained in the hydroprocessing product mixture 44.

The hydroprocessing product mixture 44 can be a complex mixture of compounds arising from hydroprocessing reaction 24, including catalytic reduction 60, catalytic hydrodeoxygenation 62, and/or catalytic hydrodeoxygenation/dehydrogenation 64 described further in FIG. 11.

After the hydroprocessing product mixture 44 may be formed, separation of the volatile and non-volatile components of the hydroprocessing product mixture 44 may take place in a volatile product mixture distillation 46 step. Conditions for distillation of such chemicals are well documented in the literature and serve as the basis for purification of many of the same chemicals from petroleum today. The volatile product mixture from the volatile product mixture distillation 46 may then advance toward lignin biobased chemicals like mixed cresolic products 50, mixed toluenic products 52, lignin biobased cresols 56, and/or lignin biobased toluenes 58 as well as lignin biofuels 28.

The volatile component of the hydroprocessing product mixture 44 obtained from the hydroprocessing reaction 24 may be treated in a number of manners including, but not limited to:
1. A volatile product mixture distillation 46 that may provide the lignin biofuels 28; or
2. A volatile product mixture distillation 46 that may be followed by a fractional distillation 54; or
3. An optional acid/base product partition 48 that may separate the product stream of volatile product mixture distillation 46 into a mixed cresolic products 50 and mixed toluenic products 52;
4. A pH adjustment/solvent partitioning process (not illustrated in FIG. 10) wherein the organic component of the hydroprocessing product mixture 44 may be first separated from a caustic reaction solution. Following concentration, the product mixture may then be subjected to any of the above above mentioned paths 1, 2, and/or 3.

The volatile product mixture obtained from the volatile product mixture distillation 46 can then:
1. move to fractional distillation 54 for production of lignin biobased cresols 56 and/or lignin biobased toluenes 58, or
2. move to an optional acid/base product partition 48 that separates the mixed cresolic products 50 and/or mixed toluenic products 52 before proceeding to the fractional distillation 54.

A volatile product mixture distillation may provide a distillate that when collected within certain boiling point ranges may yield a lignin biofuels 28 suitable for use in transportation fuels, fuel additives, and/or heating fuels. Lignin biofuels 28 may be blends of at least one or more chemicals of the lignin biobased chemicals II 26, which have physical characteristics of similar boiling point range, research octane number of about at least 90, and carbon and hydrogen content of at least about 80%. Transportation fuels and fuel additives may include automotive fuels, truck fuels, ship fuels and aircraft fuels. Heating fuels may include home heating fuels, commercial heating fuels, and/or industrial boiler fuels.

An optional acid/base product partition 48 of the hydroprocessing product mixture 44 and/or the distillate mixture from the volatile product mixture distillation 46 may permit separation of the acidic mixed cresolic products 50 from the mixed toluenic products 52. The mixed toluenic products may be hydrocarbons and non-acidic/non-basic in character. The mixed cresolic products 50 may be acidic due to the presence of the phenol group.

Alternatively, the volatile product of a volatile product mixture distillation 46 may move directly to a fractional distillation 54. The fractional distillation 54 may then provide a separation of the volatile product mixture in lignin biobased cresols 56 and/or lignin biobased toluenes 58. Moreover, and again not illustrated specifically in FIG. 10, the fractional distillation 54 may lead to a lignin biofuels 28 with a well-defined boiling point range, and/or research octane number, and/or carbon and hydrogen content.

Fractional distillation 54 of the mixed cresolic products may permit the separation of at least one chemical of the lignin biobased cresols 56, as well as performance chemical blends of at least two chemicals of lignin biobased cresols 56. The catalytic hydrodeoxygenation 62 and/or catalytic hydrodeoxygenation/dehydrogenation 64 processing may provide at least one chemical of general molecular structure:

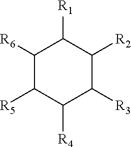

wherein R₁ is selected from among hydrogen, hydroxyl, and methoxy;
wherein R₂, R₃, R₄, R₅, and R₆ are selected from among hydrogen, methoxy, methyl, ethyl, propyl, isopropyl, butyl, isobutyl, and t-butyl; and
wherein unsaturation can exist in at least one of said products of said catalytic hydrodeoxygenation 62 and/or catalytic hydrodeoxygenation/dehydrogenation 64 processing.

The catalytic hydrodeoxygenation 62 and/or catalytic hydrodeoxygenation/dehydrogenation 64 processing 64 of FIG. 11 may provide at least one chemical of general molecular structure:

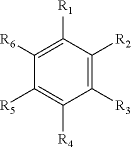

wherein R₁ is selected from among hydrogen, hydroxyl, and methoxy; and
wherein R₂, R₃, R₄, R₅, and R₆ are selected from among hydrogen, methoxy, methyl, ethyl, propyl, isopropyl, butyl, isobutyl, and t-butyl.

Fractional distillation 54 of the mixed toluenic products 52 may permit the separation of at least one chemical of the lignin biobased toluenes 58, as well as performance chemical blends of at least two chemicals of the lignin biobased toluenes 58.

The lignin biobased cresols 56 may include at least one chemical of 4-methylphenol, 3-methoxy-4-methylphenol, 2,6-dimethoxy-4-methylphenol, 2,4-dimethylphenol, 3,4-dimethyphenol, 2-methoxy-3,4-dimethylphenol, 2-methoxy-4,5-dimethylphenol, 2-methoxy-4,6-dimethylphenol, 2,6-dimethoxy-3,4-dimethylphenol, and cresols corresponding to Compounds 1-13 of FIG. 8.

The lignin biobased toluenes 58 may include at least one chemical of toluene, 1,2-dimethylbenzene, 1,3-dimethylbenzene, 1,4-dimethylbenzene, 1,2,3-trimethylbenzene, 1,2,4-trimethylbenzene, 1,3,5-trimethylbenzene, and toluenes corresponding to Compounds 1-13 of FIG. 8.

The product mix of lignin biobased cresols 56 and lignin biobased toluenes 58 may reflect the H:G:S ratio of the lignin 16. This ratio may be affected by plant species and lignin pre-treatment method even within a particular plant species. The H:G:S ratio may be controlled in part by selection of lignin feedstock (i.e., the plant species and/or pre-treatment method used to produce the lignin) and/or by blending of lignin feedstock from different plant species.

FIG. 11 details the catalytic pathways within the hydroprocessing reaction 24. The hydroprocessing of aryl carboxylic acids 36 and/or aryl aldehydes 34 from α-β lignin oxidative depolymerisation methods claimed herein may involve at least two stages:
1. Catalytic reduction 60 of the carbonyl group of the aryl carboxylic acids 36 and/or aryl aldehydes 34. This processing may convert the carboxylic acid or aldehyde group into a hydroxymethyl and/or a methyl group, and
2. Catalytic hydrodeoxygenation 62 and/or catalytic hydrodeoxygenation/dehydrogenation of the oxygen functionality on the phenyl rings of the aryl carboxylic acids 36 and/or aryl aldehydes 34. This processing may replace the methoxy groups and/or the hydroxy group on the phenyl ring, as well as any oxygen atoms on the side chain, with a hydrogen atom.

For the hydroprocessing reaction 24, there can be three potential methods of hydroprocessing: 1) catalytic reduction 60, and/or catalytic hydrodeoxygenation 62, and/or catalytic hydrodeoxygenation/dehydrogenation 64. These methods can be performed individually on lignin or sequentially to modify the final product distribution. These methods may also be repeated. As mentioned above in FIGS. 5 and 10 the degree at which the hydroprocessing reaction 24 is conducted may impact the chemical distribution of the hydroprocessing product mixture 44.

Catalytic reduction 60 may be a milder method of hydroprocessing. This process may reduce the carboxylic acid or aldehyde group present in the aryl carboxylic acids 36 and/or aryl aldehydes 34, respectively. Catalytic reduction 60 may provide entry to certain lignin biobased cresols 56.

Catalytic hydrodeoxygenation 62 is a more extensive form of hydroprocessing. The purpose of this processing may be to reductively remove a hydroxy group from a hydroxymethyl side chain (obtained from a catalytic reduction 60 step) and/or the hydroxy and/or methoxy groups on the phenyl ring of aryl carboxylic acids 36 and/or aryl aldehydes 34. Catalytic hydrodeoxygenation 62 may provide a path to lignin biobased cresols 56, and/or lignin biobased toluenes 58, and/or lignin biofuels 28.

Catalytic hydrodeoxygenation/dehydrogenation 64 may be a very extensive form of hydroprocessing. In this process, the aryl carboxylic acids 36 and/or aryl aldehydes 34 may be reduced to the level of methylcycloalkenols and/or methylcycloalkanols, which intermediates may be subsequently dehydrated and thence dehydrogenated to produce lignin biobased toluenes 58 and/or lignin biofuels 28.

These catalytic processes may be performed in any order, and as a single stage or dual stage process. Moreover, these processes may be conducted in batch or flow mode. Reaction temperatures for the hydroprocessing reaction 24 may be from about 50° C. to about 500° C. The reaction may also be conducted at a temperature of about 50° C. to about 300° C.

The hydroprocessing reaction 24 may use a metal catalyst. The catalyst may be a homogeneous species, a heterogeneous species, or a mixed metal system, or a metal species supported on an inert solid matrix. Metal catalysts may include, but are not limited to, salts and complexes of Periodic Table Group 3 through Group 12 transition metals, and/or lanthanides, and/or actinides, as well as mixed metal systems thereof. In addition, systems based on certain Group III through Group V elements of the periodic table may serve as catalyst systems. Even more specifically, some hydroprocessing catalysts may include, but are not limited to: (i) alumina supported sulfide molybdenum catalysts, (ii) zirconia and sulfated zirconia supported noble metal and bimetallic catalysts, (iii) boron-promoted bimetallic catalysts, (iv) transition metal phosphides, (v) transition meal carbides, and (vi) bifunctional zeolite supported noble metal catalysts.

Dehydration of the intermediary methyl cycloalkenols and methyl cycloalkanols may take place as part of, or subsequent to, a catalytic hydrodeoxygenation/dehydrogenation 64 step. Such dehydration may be catalysed by metals and/or the solid support such as, but not limited to zeolite catalysts, clay catalysts, and alumina catalysts. Subsequent dehydrogenation may be performed over a noble metal catalyst system. The reducing agent for the hydroprocessing reaction 24 may be hydrogen or another hydrogen source such as formate or bicarbonate. A caustic solution may be optionally used in the hydroprocessing reaction 24 of aryl carboxylic acids 36 and/or aryl aldehydes 34. The caustic used in the production of lignin biobased chemicals II 26 and/or lignin biofuels 28 may be at least one of lithium hydroxide, sodium hydroxide, potassium hydroxide, cesium hydroxide, magnesium hydroxide, barium hydroxide, and/or calcium hydroxide. This caustic may also be carbonates and/or oxides of Group I and Group II metals of the Periodic Table.

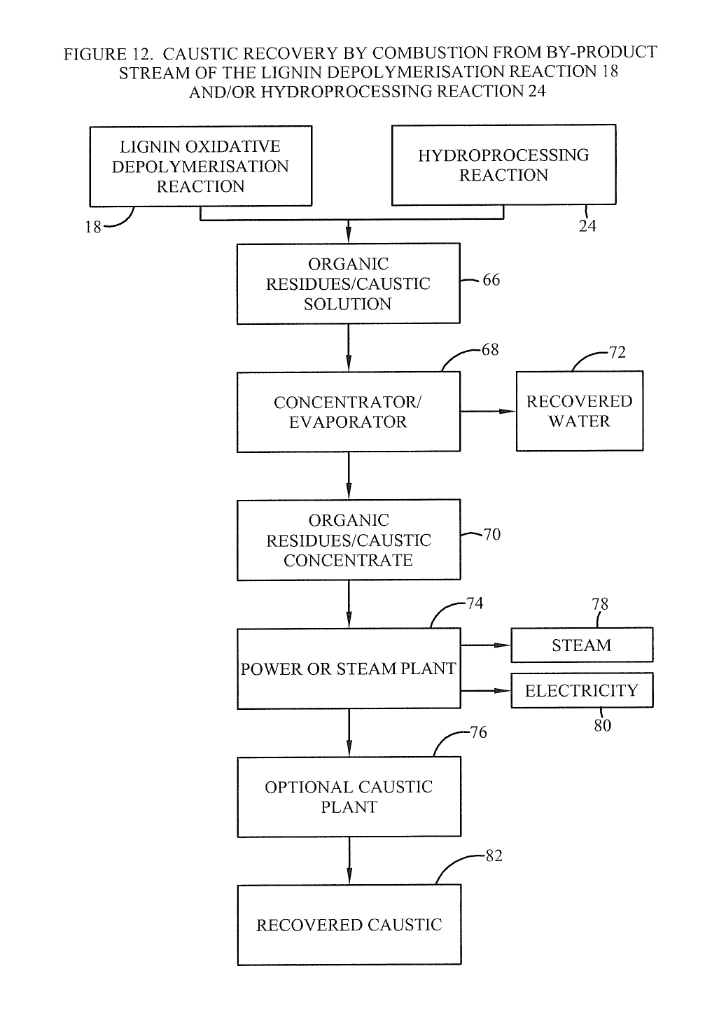

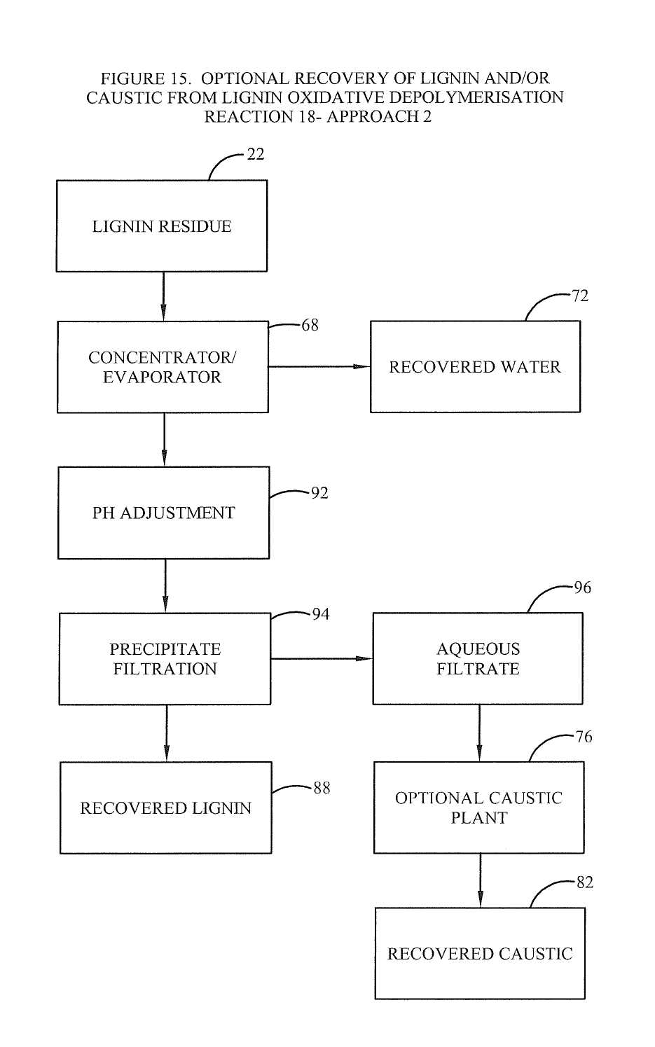

FIG. 12 shows the recovered caustic 82 and/or recovered water 72 from the hydroprocessing reaction 24 and/or lignin oxidative depolymerization reaction 18. The separation of the organic residue/caustic solution 66 from the hydroprocessing reaction 24 and/or lignin oxidative depolymerization reaction 18 can be achieved by any number of means including but not limited to: 1) concentration/evaporator 68 (detailed in this figure and in FIG. 15), and/or 2) size exclusion membrane filtration 86 (detailed in FIG. 14). The hydroprocessing reaction 24 can provide the hydroprocessing product mixture 44 as shown in FIG. 10. In addition, the lignin oxidative depolymerisation reaction 18 providing a lignin biobased chemicals I 20, a hydroprocessing product mixture 44, may be produced from the hydroprocessing reaction 24. When the reaction is conducted in a caustic solution, this lignin residue 22 may be in the form of an organic residues/caustic solution 66. Moreover, in addition to providing a lignin biobased chemicals II 26 and/or lignin biofuels 28 from the hydroprocessing reaction 24, a by-product stream organic residues/caustic solution 66 may also be formed.

In order to enhance the greenness of this method, and to reduce waste expenses and raw material costs, it may be important to recover/recycle the caustic from these by-product streams. This recovered caustic 82 may be lithium hydroxide, sodium hydroxide, potassium hydroxide, cesium hydroxide, magnesium hydroxide, barium hydroxide, and/or calcium hydroxide. This recovered caustic 82 may also be carbonates and/or oxides of Group I and Group II metals of the Periodic Table. In addition, the recovery of the caustic may provide for steam 78 and/or electricity 80 generation by combustion of the organic residues. To achieve this caustic recovery, the organic residues/caustic solution 66 may be concentrated on a concentrator/evaporator 68 to give recovered water 72 and an organic residues/caustic concentrate 70. This reduction in water content can be considered as an optional step; however, it generally assists in getting complete combustion of the organic residues in the power or steam plant 74 step. The combustion of organic residues/caustic concentrate 70 in the power or steam plant 74 may provide steam 78 and/or electricity 80. Combustion may be also beneficial in that it may be considered to provide zero CO₂ emissions. In this regard, the organic residues of lignin processing to biobased chemicals and biofuels may serve as a form of combustible fuel for energy production, wherein such energy may be heat or electricity. The recovery of these caustic forms may then take place post combustion of the organic residues, followed by dissolution of the combustion pot residues into water and thence removal of any insoluble combustion ash by filtration. The pot residues from combustion process of the organic residues/caustic concentrate 70 may be comprised of the caustic and organic ash. These pot residues may be sent to an optional caustic plant 76 for regeneration of the recovered caustic 82. The recovered caustic 82 may then be recycled to the lignin oxidative depolymerisation reaction 18.

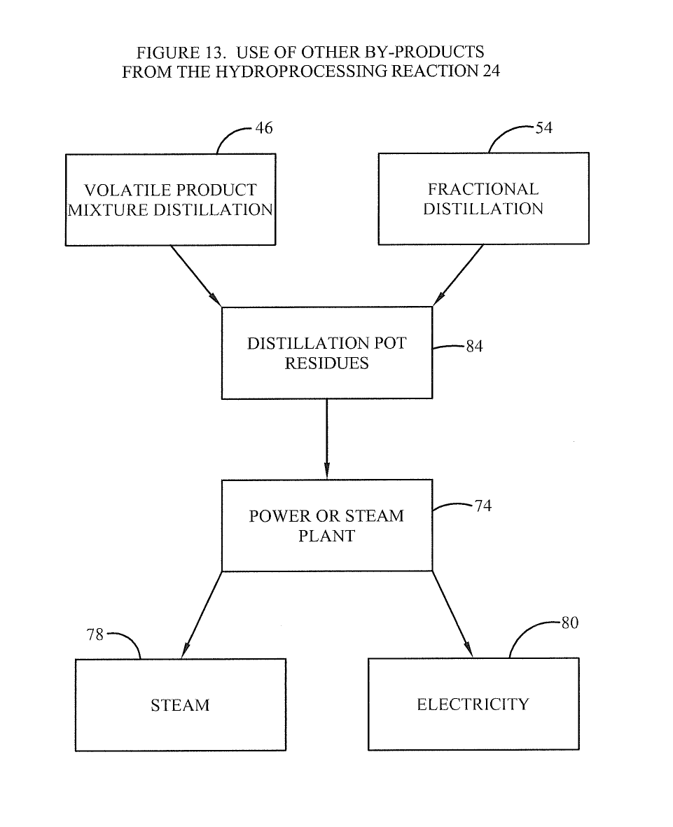

FIG. 13 depicts additional ways in which certain other organic by-products of the hydroprocessing reaction 24 may serve as fuel equivalents to maximize utilization and value from lignin biomass. As seen previously in FIG. 10, the hydroprocessing reaction 24 may provide valuable biobased products such as lignin biofuels 28, lignin biobased cresols 56, and lignin biobased toluenes 58. FIG. 13 now illustrates additional pathways that may extract maximal value from the remaining by-products of lignin processing. First, the volatile product mixture distillation 46 of the hydroprocessing product mixture 44 may yield a distillation pot residues 84 that may serve as a combustible fuel equivalent. Secondly, fractional distillation 54 of the mixed cresolic products 50, and/or the mixed toluenic products 52, and/or the distillate from volatile product mixture distillation 46 may yield a distillation pot residues 84 that may serve as a combustible fuel equivalent. These distillation pot residues 84 may be sent to a power or steam plant 74 wherein they may be combusted as fuel to produce energy. This energy produced from combustion of the distillation pot residues may be in the form of steam 78 and/or electricity 80.

FIG. 14 provides a method for recovering recovered lignin 88 and/or recovered caustic 82 through the use of size exclusion membrane filtration 86.

Lignin may be a valuable source of polyols for polymer and resin production or for use in the production of other biobased chemicals and fuels as described A METHOD FOR PRODUCING BIOBASED CHEMICALS FROM PLANT LIGNIN (U.S. application Ser. No. 13/453,422 filed Apr. 23, 2012). As such, it may be economically beneficial to be able to recover lignin from the lignin residue 22 of a lignin oxidative depolymerisation reaction 18. Moreover, to increase the greenness of the method claimed herein, and to reduce waste treatment expenses and raw material costs, the optional recovery of caustic may be important when caustic is employed in the lignin oxidative depolymerisation reaction 18.

One approach to recover lignin from a reaction by-product solution may be the use of size exclusion membrane filtration 86. In this process, the lignin residue 22 may not be able to pass through the membrane, allowing for separation of a solid recovered lignin 88 away from the aqueous solution in which the lignin residue 22 may be soluble. If caustic is employed in the lignin oxidative depolymerisation reaction 18, the aqueous solution obtained by size exclusion membrane filtration 86 may be a recovered caustic solution 90. The recovered lignin 88 may be a neutral lignin or a metal phenolate. For example, this process may provide a sodium phenolate, or potassium phenolate, or calcium phenolate of the recovered lignin 88, respectively, if sodium hydroxide, or potassium hydroxide, or calcium hydroxide may be used as the caustic in the lignin oxidative depolymerisation reaction 18. The recovered caustic solution 90 may in turn move to the optional caustic plant 76 for recovery of the caustic. The recovered caustic 82 may then be recycled to an additional lignin oxidative depolymerisation reaction 18.

FIG. 15 provides yet another optional method for providing recovered lignin 88 and/or recovered caustic 82. Like the method for recovering recovered lignin 88 and/or recovered caustic 82 through the use of size exclusion membrane filtration 86 in FIG. 14, a pH precipitation of the lignin done through a pH adjustment 92 step may also be used. The pH adjustment may lead to precipitation of the lignin. The pH adjustment 92 step may optionally follow an optional concentration of the aqueous solution of lignin on a concentrator/evaporator 68.

The recovered lignin 88 formed by pH induced precipitation may be separated from the aqueous filtrate 96 by a precipitate filtration 94 step. The recovered lignin 88 obtained herein may have a reduced metal ion content relative to that recovered lignin 88 obtained in the approach outlined in FIG. 14. The aqueous filtrate 96 may then transfer to the optional caustic plant 76 for recovery of the caustic. The process for caustic regeneration herein may differ from that previously described in FIGS. 12 and 14, since the aqueous filtrate may contain inorganic salts from acid neutralization of the caustic. The recovered caustic 82 may then be recycled back into the lignin oxidative depolymerisation reaction 18.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

1. A method for biorefining, comprising the steps of:
providing lignin biomass;
processing said lignin biomass; and
producing at least one product from said lignin biomass.

2. The method of claim 1, wherein said lignin biomass is comprised of at least one lignin building block of p-coumaryl alcohol, coniferyl alcohol, and sinapyl alcohol.

3. The method of claim 1, wherein said lignin biomass is provided from at least one biomass of plant biomass, woody plant biomass, agricultural plant biomass, and cultivated plant biomass.

4. The method of claim 1, wherein said lignin biomass is provided from at least one biomass of fresh plant biomass, recovered plant biomass, pulp and paper mill biomass, cellulosic ethanol refinery biomass, sugar cane mill biomass, commercial plant biomass fractionator biomass, and lignin residue biomass.

5. The method of claim 1, wherein said lignin biomass is provided from kraft pulp mill lignin.

6. The method of claim 1, wherein said lignin biomass is provided from sulfite pulp mill lignin.

7. The method of claim 1, wherein said lignin biomass is provided from soda pulp mill lignin.

8. The method of claim 1, wherein said lignin biomass is provided from cellulosic ethanol refinery lignin.

9. The method of claim 1, wherein said lignin biomass is provided from commercial plant biomass fractionator lignin.

10. The method of claim 1, wherein said lignin biomass is provided from lignin residue lignin.

11. The method of claim 1, wherein said lignin biomass is provided from waste lignin.

12. The method of claim 9, wherein said waste lignin is provided from at least one waste lignin of recovered biomass, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin.

13. The method of claim 1, further comprising the step of:
providing a lignin pretreatment to said lignin biomass.

14. The method of claim 1, wherein said processing of said lignin biomass is provided from at least one process of chemical-induced processing, catalytic oxidative lignin depolymerisation processing, and catalytic hydroprocessing.

15. The method of claim 14, wherein said chemical-induced processing is provided from at least one process of oxidative lignin depolymerisation processing and caustic-induced lignin depolymerisation processing.

16. The method of claim 15, wherein said chemical-induced processing uses an oxidant.

17. The method of claim 16, wherein said oxidant comprises at least one oxidant of air, oxygen, hydrogen peroxide, hydrogen peroxide, organic peroxide, and organic nitro compound.

18. The method of claim 15, wherein said chemical-induced processing is controlled for selecting at least one of said products from said lignin biomass.

19. The method of claim 15, wherein said chemical-induced processing is performed at a reaction temperature of about 50° C. to about 500° C.

20. The method of claim 14, wherein said chemical-induced processing is performed at a reaction temperature of about 80° C. to about 350° C.

21. The method of claim 14, wherein said chemical-induced processing is performed at a reaction temperature of about 100° C. to about 250° C.

22. The method of claim 14, wherein said chemical-induced processing is induced by caustic.

23. The method of claim 22, wherein said caustic is comprised of at least one caustic of lithium hydroxide, sodium hydroxide, potassium hydroxide, cesium hydroxide, magnesium hydroxide, barium hydroxide, calcium hydroxide, and carbonates and oxides of Group I and Group II metals of the Periodic Table.

24. The method of claim 14, wherein said catalytic oxidative lignin depolymerisation processing of said lignin biomass and at least one of said products of said lignin biomass is provided from at least one catalyst of a metal salt, a metal complex, and an elemental metal.

25. The method of claim 24 wherein said catalyst used in said catalytic oxidative lignin depolymerisation processing is provided from at least one catalyst of Group 3 through Group 12 transitional elements of the Periodic Table.

26. The method of claim 24 wherein said catalyst of said catalytic oxidative lignin depolymerisation processing is at least one catalyst type of homogeneous catalyst, heterogeneous catalyst, and supported catalyst on a solid matrix.

27. The method of claim 14, wherein said catalytic oxidative lignin depolymerisation processing provides non-selective oxidation of said lignin biomass or at least one of said products of said lignin biomass.

28. The method of claim 14, wherein said catalytic oxidative lignin depolymerisation processing provides selective oxidation of said lignin biomass and at least one of said products of said lignin biomass.

29. The method of claim 24, wherein an oxidant is provided for said catalytic oxidative lignin depolymerisation processing and is selected from at least one oxidant of air, oxygen, hydrogen peroxide, hydrogen peroxide, organic peroxide, and organic nitro compound.

30. The method of claim 24, wherein said catalytic oxidative lignin depolymerisation processing is conducted at a reaction temperature of about 50° C. to about 300° C.

31. The method of claim 24, wherein said catalytic oxidative lignin depolymerisation processing is conducted at a reaction temperature of about 100° C. to about 200° C.

32. The method of claim 24, wherein said catalytic oxidative lignin depolymerisation processing provides at least one of said products retaining at least 66% of the original carbon atom structure of said lignin biomass.

33. The method of claim 24, wherein said catalytic oxidative lignin depolymerisation processing provides at least one of said products retaining at least 77% of the carbon atom structure of said lignin biomass.

34. The method of claim 24, wherein said catalytic oxidative lignin depolymerisation processing provides at least one of said products retaining at least 88% of the carbon atom structure of said lignin biomass.

35. The method of claim 24, wherein said catalytic oxidative lignin depolymerisation processing provides at least one of said products retaining 100% of the carbon atom structure of said lignin biomass.

36. The method of claim 24, wherein said lignin biomass has a weight, and said catalytic oxidative lignin depolymerisation processing provides lignin residues having a weight of about 10% to about 90% of said lignin biomass weight.

37. The method of claim 24, wherein said lignin biomass has a weight, and said catalytic oxidative lignin depolymerisation processing provides lignin residues having a weight of about 10% to about 50% of said lignin biomass weight.

38. The method of claim 14, wherein said catalytic hydroprocessing provides non-selective reduction of said lignin biomass and said products of said lignin biomass.

39. The method of claim 14, wherein said catalytic hydroprocessing provides selective reduction of said lignin biomass and said products of said lignin biomass.

40. The method of claim 14, wherein said catalytic hydroprocessing of said lignin biomass and said products of said lignin biomass is provided by at least one process of catalytic reduction processing, catalytic hydrodeoxygenation processing, and catalytic hydrodeoxygenation/dehydrogenation processing.

41. The method of claim 40, wherein said catalytic reduction processing, said catalytic hydrodeoxygenation processing, and said catalytic hydrodeoxygenation/dehydrogenation processing of said lignin biomass and said products of said lignin biomass is provided in any order.

42. The method of claim 40, wherein said catalytic reduction processing, said catalytic hydrodeoxygenation processing, and said catalytic hydrodeoxygenation/dehydrogenation processing of said lignin biomass and said products of said lignin biomass is provided by single stage processing or dual stage processing.

43. The method of claim 40, wherein said catalytic hydrodeoxygenation processing further comprise the steps of:
processing using catalytic hydrodeoxygenation; and
processing using catalytic dehydrogenation.

44. The method of claim 40, wherein said catalytic hydrodeoxygenation/dehydrogenation processing further comprises the step of:
processing using catalytic dehydration.

45. The method of claim 44, wherein the catalyst of said catalytic dehydration processing is provided by at least one catalyst of zeolite type catalysts, clay catalysts, and alumina support catalysts.

46. The method of claim 40, wherein said catalytic hydrodeoxygenation processing and catalytic hydrodeoxygenation/dehydrogenation processing provides at least one chemical of general molecular structure: 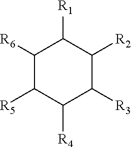
wherein R₁ is selected from among hydrogen, hydroxyl, and methoxy;
wherein R₂, R₃, R₄, R₅, and R₆ are selected from among hydrogen, methoxy, methyl, ethyl, propyl, isopropyl, butyl, isobutyl, and t-butyl; and
wherein unsaturation can exist in at least one of said products of said catalytic hydrodeoxygenation processing.

47. The method of claim 40, wherein said catalytic/dehydrogenation processing and catalytic hydrodeoxygenation/dehydrogenation processing provides at least one chemical of general molecular structure: 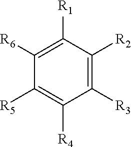
wherein R₁ is selected from among hydrogen, hydroxyl, and methoxy; and
wherein R₂, R₃, R₄, R₅, and R₆ are selected from among hydrogen, methoxy, methyl, ethyl, propyl, isopropyl, butyl, isobutyl, and t-butyl.

48. The method of claim 14, wherein said catalytic hydroprocessing of said lignin biomass and at least one of said products of said lignin biomass is performed at a reaction temperature of about 50° C. to about 500° C.

49. The method of claim 14, wherein said catalytic hydroprocessing of said lignin biomass and at least one of said products of said lignin biomass is performed at a reaction temperature of about 50° C. to about 300° C.

50. The method of claim 14, wherein said catalytic hydroprocessing uses at least one catalyst provided from Group 3 through Group 12 transitional elements of the Periodic Table.

51. The method of claim 14, wherein said catalytic hydroprocessing uses at least one catalyst provided from Group III through Group V elements of the Periodic Table.

52. The method of claim 14, wherein said catalytic hydroprocessing uses a reducing agent provided by at least one reducing agent of hydrogen and hydrogen-donating liquids.

53. The method of claim 1, wherein said processing of said lignin biomass is provided from at least one process of batch processing and flow processing.

54. The method of claim 1, wherein said processing of said lignin biomass is conducted in caustic provided by at least one caustic of lithium hydroxide, sodium hydroxide, potassium hydroxide, cesium hydroxide, magnesium hydroxide, barium hydroxide, calcium hydroxide, and carbonates and oxides of Group I and Group II metals of the Periodic Table.

55. The method of claim 1, wherein said processing of said lignin biomass is conducted in solvent provided by at least one solvent of water, ethanol, propanol, isopropanol, acetonitrile, and ionic liquids.

56. The method of claim 1, wherein at least one of said products from said lignin biomass comprises at least one product of biobased chemicals, biofuels, and lignin residues.

57. The method of claim 1, wherein at least one of said products from said lignin biomass comprises at least two products of biobased chemicals, biofuels, and lignin residues.

58. The method of claim 56, wherein said biobased chemicals comprise at least one chemical of commodity chemicals, fine chemicals, and specialty chemicals.

59. The method of claim 56, wherein said biobased chemicals comprise at least one chemical of achiral chemicals, racemic chemicals, and chiral chemicals.

60. The method of claim 2, wherein a ratio of said lignin building blocks provides control of a composition of at least one of said products from said lignin biomass.

61. The method of claim 2, wherein a ratio of said lignin building blocks provides control of a composition of at least two of said products from said lignin biomass.

62. The method of claim 56, wherein said biobased chemicals comprise at least one chemical of aryl aldehydes, aryl carboxylic acids, aryl ketones, and aliphatic carboxylic acids.

63. The method of claim 56, wherein said biobased chemicals comprise at least two chemicals of aryl aldehydes, aryl carboxylic acids, aryl ketones, alkyl carboxylic acids.

64. The method of claim 62, wherein said at least one chemical of aryl aldehydes, aryl carboxylic acids, aryl ketones, and aliphatic carboxylic acids are provided by catalytic oxidative lignin depolymerisation processing.

65. The method of claim 62, wherein said aryl aldehydes comprise at least one chemical of 4-hydroxybenzaldehyde, vanillin, and syringaldehyde.

66. The method of claim 62, wherein said aryl aldehydes comprise at least one chemical of (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, and 4-hydroxy-3,5-dimethoxycinnaminaldehyde.

67. The method of claim 62, wherein said aryl carboxylic acids comprise at least one chemical of 4-hydroxybenzoic acid, vanillic acid, and syringic acid.

68. The method of claim 62, wherein said aryl carboxylic acids comprise at least one chemical of general molecular structure: 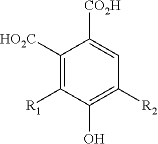
wherein R₁ and R₂ are selected from among hydrogen and methoxy.

69. The method of claim 62, wherein said aryl carboxylic acids comprise at least one chemical of general molecular structure: 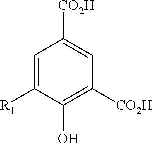
wherein R₁ is selected from among hydrogen and methoxy.

70. The method of claim 62, wherein said aryl carboxylic acids comprise at least one chemical of general molecular structure: 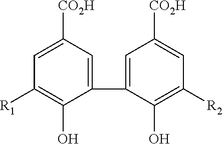
wherein R₁ and R₂ are selected from among hydrogen and methoxy.

71. The method of claim 62, wherein said aryl carboxylic acids comprise at least one chemical of general molecular structure: 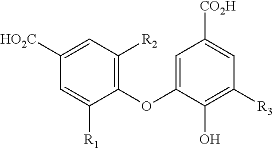
wherein R₁, R₂, and R₃ are selected from among hydrogen and methoxy.

72. The method of claim 62, wherein said aryl carboxylic acids comprise at least one chemical of (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

73. The method of claim 62, wherein said aryl aldehydes and said aryl carboxylic acids comprise at least one chemical of 4-hydroxybenzaldehyde, vanillin, syringaldehyde, 4-hydroxybenzoic acid, vanillic acid, and syringic acid.

74. The method of claim 62, wherein said aryl ketones comprise at least one chemical of 1-(4-hydroxyphenyl)ethanone, 1-(4-hydroxy-3-methoxyphenyl)ethanone, and 1-(4-hydroxy-3,5-dimethoxyphenyl)ethanone.

75. The method of claim 62, wherein said aryl ketones comprise at least one chemical of 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1-propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, and 1-(4-hydroxy-3,5-dimethoxyphenyl)-2-propanone.

76. The method of claim 62, wherein said aliphatic carboxylic acids comprise at least one chemical of formic acid, oxalic acid, acetic acid, glycolic acid, glyoxylic acid, propionic acid, lactic acid, and malonic acid.

77. The method of claim 56, wherein said biobased chemicals comprise at least one chemical of phenols, alkyl phenols, alkenyl phenols, and performance chemicals.

78. The method of claim 56, wherein said biobased chemicals comprise at least two chemicals of phenols, alkyl phenols, alkenyl phenols, and performance chemicals.

79. The method of claim 77, wherein said at least one chemical of phenols, alkyl phenols, alkenyl phenols, and performance chemicals are provided by catalytic hydroprocessing.

80. The method of claim 77, wherein said phenols comprise at least one chemical of phenol, guaiacol, and 2,6-dimethoxyphenol.

81. The method of claim 77, wherein said alkyl phenols comprise at least one chemical of 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, and 2,4,6-trimethylphenol.

82. The method of claim 77, wherein said alkyl phenols comprise at least one chemical of a general molecular structure: 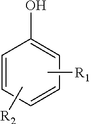
wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₁ and R₂ are located at positions 2, 3, 4, or 5 of the phenol ring.

83. The method of claim 77, wherein said alkyl phenols comprise at least one chemical of a general molecular structure: 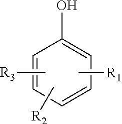
wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

84. The method of claim 77, wherein said alkyl phenols comprise at least one chemical of 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, and 2,6-dimethoxy-4-t-butylphenol.

85. The method of claim 77, wherein said alkyl phenols comprise at least one chemical of general molecular structure: 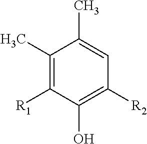
wherein R₁ and R₂ are selected from among hydrogen and methoxy.

86. The method of claim 77, wherein said alkyl phenols comprise at least one chemical of general molecular structure: 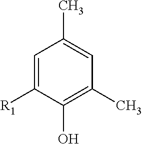
wherein R₁ is selected from among hydrogen and methoxy.

87. The method of claim 77, wherein said alkyl phenols comprise at least one chemical of general molecular structure: 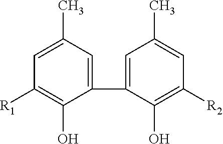
wherein R₁ and R₂ are selected from among hydrogen and methoxy.

88. The method of claim 77, wherein said alkyl phenols comprise at least one chemical of general molecular structure: 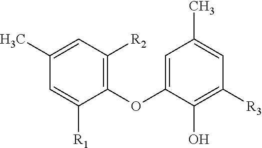
wherein R₁, R₂, and R₃ are selected from among hydrogen and methoxy.

89. The method of claim 77, wherein said alkenyl phenols comprise at least one chemical of 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and iso-syringeugenol.

90. The method of claim 77, wherein said performance chemicals comprise at least one chemical of products comprising said phenols, said alkyl phenols, and said alkenyl phenols.

91. The method of claim 56, wherein said biobased chemicals comprise at least one chemical of benzene, toluene, xylenes, mesitylenes, biaryls, aryl alkanes, aryl alkenes, alkanes, alkenes, cycloalkanes, cycloalkenes, alkyl esters, and performance chemicals.

92. The method of claim 56, wherein said biobased chemicals comprise at least two chemicals of benzene, toluene, xylenes, mesitylenes, biaryls, aryl alkanes, aryl alkenes, alkanes, alkenes, cycloalkanes, cycloalkenes, alkyl esters, and performance chemicals.

93. The method of claim 91, wherein said at least one chemical of benzene, toluene, xylenes, mesitylenes, biaryls, aryl alkanes, aryl alkenes, alkanes, alkenes, cycloalkanes, cycloalkenes, alkyl esters, and performance chemicals are provided by catalytic hydroprocessing.

94. The method of claim 91, wherein said biobased chemicals comprise at least one chemical of benzene, toluene, 1,2-dimethylbenzene, 1,3-dimethylbenzene, 1,4-dimethylbenzene, 1,2,3-trimethylbenzene, 1,2,4-trimethylbenzene and 1,3,5-trimethylbenzene.

95. The method of claim 91, wherein said biaryls comprise at least chemical of biphenyl, 4,4′-dimethylbiphenyl, 3,3′-dimethylbiphenyl, 2,2′-dimethylbiphenyl, 3,4′-dimethylbiphenyl, 2,4′-dimethylbiphenyl, 2,3′-dimethylbiphenyl, 4,4′-diethylbiphenyl, 3,3′-diethylbiphenyl, 2,2′-diethylbiphenyl, 3,4′-diethylbiphenyl, 2,4′-diethylbiphenyl, 2,3′-diethylbiphenyl, 4,4′-dipropylbiphenyl, 3,3′-dipropylbiphenyl, 2,2′-dipropylbiphenyl, 3,4′-dipropylbiphenyl, 2,4′-dipropylbiphenyl, and 2,3′-dipropylbiphenyl.

96. The method of claim 91, wherein said aryl alkanes comprise at least one chemical of ethylbenzene, propylbenzene, isopropylbenzene, butylbenzene, isobutylbenzene, and t-butylbenzene.

97. The method of claim 91, wherein said aryl alkanes comprise at least one chemical of a general molecular structure: 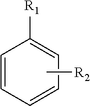
wherein R₁ is selected from among methyl, ethyl, propyl, isopropyl, butyl, isobutyl, and t-butyl;
wherein R₂ is selected from among ethyl, propyl, isopropyl, butyl, isobutyl, and t-butyl; and
wherein R₂ is located at positions 2, 3, 4, or 5 of the ring.

98. The method of claim 91, wherein said aryl alkanes comprise at least one chemical of a general molecular structure: 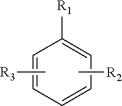
wherein R₁ and R₂ are selected from among methyl, ethyl, propyl, isopropyl, butyl, isobutyl, and t-butyl;
wherein R₃ is selected from among ethyl, propyl, isopropyl, butyl, isobutyl, and t-butyl; and
wherein R₂ and R₃ are located at positions 2, 3, 4, or 5 of the ring.

99. The method of claim 91, wherein said aryl alkenes comprise at least one chemical of styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, and 1-(4-methylphenyl)-2-propene.

100. The method of claim 91, wherein said alkanes comprise at least one chemical of hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, and 3,4,5-trimethylnonane.

101. The method of claim 91, wherein said alkenes comprise at least one compound of a partially unsaturated alkane.

102. The method of claim 91, wherein said cycloalkanes comprise at least one chemical of cyclopentane, cyclohexane, cycloheptane, methylcyclopentane, methylcyclohexane, methylcycloheptane, ethylcyclopentane, ethylcyclohexane, ethylcycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and 1,4-dimethylcycloheptane.

103. The method of claim 91, wherein said cycloalkanes comprise at least one chemical of a general molecular structure: 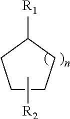
wherein n is 1, 2, or 3;
wherein R₁ is selected from among methyl, ethyl, propyl, isopropyl, butyl, isobutyl, and t-butyl;
wherein R₂ is selected from among ethyl, propyl, propyl, isopropyl, butyl, isobutyl, and t-butyl; and
wherein R₂ is located at any ring position other than that of R₁.

104. The method of claim 91, wherein said cycloalkenes comprise at least one compound of a partially unsaturated cycloalkane.

105. The method of claim 91, wherein said alkyl esters comprise at least one chemical of a general molecular structure: 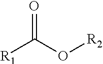
wherein R₁ and R₂ are selected from among methyl, ethyl, propyl, isopropyl, butyl, isobutyl, and t-butyl.

106. The method of claim 91, wherein said performance chemicals comprise at least one of said chemicals of benzene, toluene, xylenes, mesitylenes, aryl alkanes, aryl alkenes, alkanes, alkenes, cycloalkanes, cycloalkenes, and alkyl esters.

107. The method of claim 56, wherein said biofuels comprise at least one chemical of alkanes, alkenes, cycloalkanes, cycloalkenes, alkyl esters, benzene, toluene, xylenes, mesitylenes, biaryls, aryl alkanes, aryl alkenes, alkyl naphthalenes, phenols, alkyl phenols, and alkenyl phenols.

108. The method of claim 56, wherein said biofuels comprise blends of at least two chemicals of alkanes, alkenes, cycloalkanes, cycloalkenes, alkyl esters, benzene, toluene, xylenes, mesitylenes, biaryls, aryl alkanes, aryl alkenes, alkyl naphthalenes, phenols, alkyl phenols, and alkenyl phenols.

109. The method of claim 108, wherein said blends of said biofuels comprise product mixtures of chemicals of similar boiling point range.

110. The method of claim 108, wherein said blends of said biofuels comprise product mixtures of chemicals with a carbon and hydrogen content of about 80% to about 100%.

111. The method of claim 108, wherein said blends of said biofuels comprise product mixtures of chemicals with a research octane number of at least about 90.

112. The method of claim 108, wherein said blends of said biofuels are comprised of at least one fuel of transportation fuels, heating fuels, and fuel additives.

113. The method of claim 112, wherein said transportation fuels serve at least one market of automobile fuels, truck fuels, ship fuels, and aircraft fuels.

114. The method of claim 112, wherein said heating fuels serve at least one market of home heating fuels, commercial heating fuels, and industrial boiler fuels.

115. The method of claim 112, wherein said fuel additives serve at least one market of transportation fuels and heating fuels.

116. The method of claim 1, further comprising the step of:
using at least one product from said lignin biomass in the production of other derivative chemicals, materials, and products.

117. The method of claim 116, wherein said other derivative chemicals, materials, and products comprise at least one chemical of aryl aldehydes, aryl carboxylic acids, aryl nitriles, aryl alcohols, and aryl esters.

118. The method of claim 117, wherein said aryl aldehydes of said derivative chemicals, materials, and products comprise at least one chemical of 4-hydroxybenzaldehyde, vanillin, and syringaldehyde.

119. The method of claim 117, wherein said aryl aldehydes of said derivative chemicals, materials, and products comprise at least one chemical of general molecular structure: 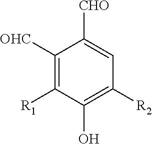
wherein R₁ and R₂ are selected from among hydrogen and methoxy.

120. The method of claim 117, wherein said aryl aldehydes of said derivative chemicals, materials, and products comprise at least one chemical of general molecular structure: 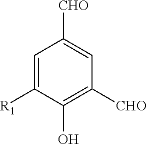
wherein R₁ is selected from among hydrogen and methoxy.

121. The method of claim 117, wherein said aryl aldehydes of said derivative chemicals, materials, and products comprise at least one chemical of general molecular structure: 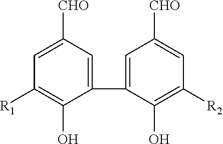
wherein R₁ and R₂ are selected from among hydrogen and methoxy.

122. The method of claim 117, wherein said aryl aldehydes of said derivative chemicals, materials, and products comprise at least one chemical of general molecular structure: 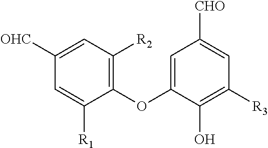
wherein R₁, R₂, and R₃ are selected from among hydrogen and methoxy.

123. The method of claim 117, wherein said aryl carboxylic acids of said derivative chemicals, materials, and products comprise at least one chemical of 4-hydroxybenzoic acid, vanillic acid, and syringic acid.

124. The method of claim 117, wherein said aryl esters of said derivative chemicals, materials, and products comprise a C₁-C₁₆ ester of at least one chemical of 4-hydroxybenzoic acid, vanillic acid, and syringic acid.

125. The method of claim 117, wherein said aryl esters of said derivative chemicals, materials, and products comprise a C₁-C₁₆ ester of at least one chemical of general molecular structure: 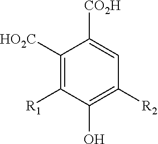
wherein R₁ and R₂ are selected from among hydrogen and methoxy.

126. The method of claim 117, wherein said aryl esters of said derivative chemicals, materials, and products comprise a C₁-C₁₆ ester of at least one chemical of general molecular structure: 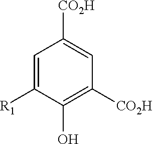
wherein R₁ is selected from among hydrogen and methoxy.

127. The method of claim 117, wherein said aryl esters of said derivative chemicals, materials, and products comprise a C₁-C₁₆ ester of at least one chemical of general molecular structure: 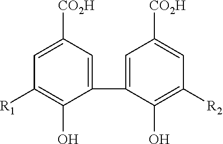
wherein R₁ and R₂ are selected from among hydrogen and methoxy.

128. The method of claim 117, wherein said aryl esters of said derivative chemicals, materials, and products comprise a C₁-C₁₆ ester of at least one chemical of general molecular structure: 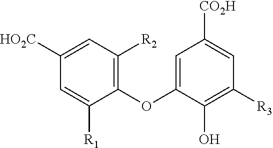
wherein R₁, R₂, and R₃ are selected from among hydrogen and methoxy.

129. The method of claim 117, wherein said aryl nitriles of said derivative chemicals, materials, and products comprise at least one chemical of 4-hydroxybenzonitrile, 4-hydroxy-3-methoxybenzonitrile, and 4-hydroxy-3,5-dimethoxybenzonitrile.

130. The method of claim 117, wherein said aryl nitriles of said derivative chemicals, materials, and products comprise at least one chemical of general molecular structure: 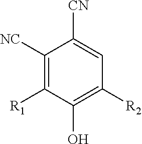
wherein R₁ and R₂ are selected from among hydrogen and methoxy.

131. The method of claim 117, wherein said aryl nitriles of said derivative chemicals, materials, and products comprise at least one chemical of general molecular structure: 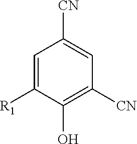
wherein R₁ is selected from among hydrogen and methoxy.

132. The method of claim 117, wherein said aryl nitriles of said derivative chemicals, materials, and products comprise at least one chemical of general molecular structure: 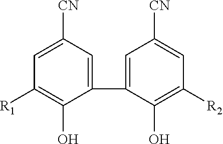
wherein R₁ and R₂ are selected from among hydrogen and methoxy.

133. The method of claim 117, wherein said aryl nitriles of said derivative chemicals, materials, and products comprise at least one chemical of general molecular structure: 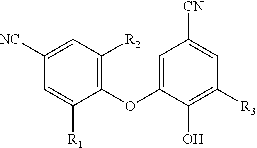
wherein R₁, R₂, and R₃ are selected from among hydrogen and methoxy.

134. The method of claim 117, wherein said aryl alcohols of said derivative chemicals, materials, and products comprise at least one chemical of 4-hydroxybenzyl alcohol, 4-hydroxy-3-methoxybenzyl alcohol, and 4-hydroxy-3,5-dimethoxybenzyl alcohol.

135. The method of claim 117, wherein said aryl alcohols of said derivative chemicals, materials, and products comprise at least one chemical of general molecular structure: 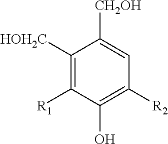
wherein R₁ and R₂ are selected from among hydrogen and methoxy.

136. The method of claim 117, wherein said aryl alcohols of said derivative chemicals, materials, and products comprise at least one chemical of general molecular structure: 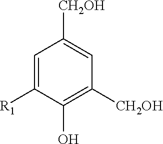
wherein R₁ is selected from among hydrogen and methoxy.

137. The method of claim 117, wherein said aryl alcohols of said derivative chemicals, materials, and products comprise at least one chemical of general molecular structure: 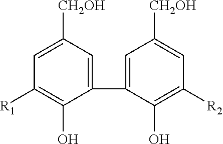
wherein R₁ and R₂ are selected from among hydrogen and methoxy.

138. The method of claim 117, wherein said aryl alcohols of said derivative chemicals, materials, and products comprise at least one chemical of general molecular structure: 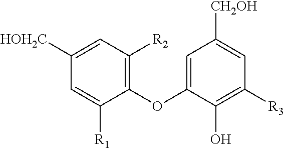
wherein R₁, R₂, and R₃ are selected from among hydrogen and methoxy.

139. The method of claim 56, wherein said lignin residues provide energy production.

140. The method of claim 139, wherein said energy production is heat or power.

141. The method of claim 56, wherein said lignin residue is subjected to further processing to produce at least one additional product.

142. The method of claim 1, wherein said lignin biomass has a weight, and a waste product of said lignin biomass is less than 30% of said lignin biomass weight.

143. The method of claim 1, wherein said lignin biomass has a weight, and a waste product of said lignin biomass is less than 20% of said lignin biomass weight.

144. The method of claim 1, wherein said lignin biomass has a weight, and a waste product of said lignin biomass is less than 10% of said lignin biomass weight.

145. The method of claim 1, wherein waste products of said processing of said lignin biomass provide energy production.

146. The method of claim 145, wherein said energy production is heat or power.

147. The method of claim 1, further comprising the step of:
recovering and recycling caustic from said processing of said lignin biomass.

148. The method of claim 147, wherein size exclusion membrane filtration is used for said recovering and recycling caustic from said processing of said lignin biomass.

149. The method of claim 147, wherein a pH precipitation is used for said recovering and recycling caustic from said processing of said lignin biomass.

150. The method of claim 1, further comprising the step of functionalizing said lignin biomass prior to said producing at least one of said products from said lignin biomass.

151. The method of claim 1, wherein said product of said lignin biomass has an economic value higher than boiler fuel.

152. The method of claim 1, wherein said processing of said lignin biomass produces at least two products of differing economic value.

153. The method of claim 1, wherein selective production of said product from said lignin biomass occurs.

154. A method for biorefining, comprising the steps of:
providing lignin biomass comprising at least one biomass of woody plant biomass, agricultural plant biomass, cultivated plant biomass, kraft pulping biomass, sulfite pulping biomass, soda pulping biomass, cellulosic ethanol refinery biomass, sugar cane mill biomass, lignin residue biomass, and waste biomass;
processing said lignin biomass with chemical-induced processing provided by chemical-induced processing, catalytic oxidative lignin depolymerisation processing, and catalytic hydroprocessing;
processing said lignin biomass with catalytic hydroprocessing from at least one process of catalytic reduction processing, catalytic hydrodeoxygenation processing, and catalytic/dehydrogenation processing;
processing of said lignin biomass from at least one catalytic process to selectively provide at least one product which retains at least 77% of the carbon atom structure of said lignin biomass;
functionalizing said lignin biomass prior to producing at least one product from said lignin biomass;
producing at least one product from said lignin biomass comprising at least one product of biobased chemicals, biobased fuels, and lignin residues;
producing a plurality of products from said lignin biomass comprising at least one chemical of aryl aldehydes, aryl carboxylic acids, aryl ketones, aliphatic carboxylic acids, phenols, alkyl phenols, alkenylphenols, benzene, toluene, xylenes, mesitylenes, biaryls, aryl alkanes, aryl alkenes, alkanes, alkenes, cycloalkanes, cycloalkenes, alkyl esters, and performance chemicals;
reducing the waste product of said lignin biomass, wherein said lignin biomass has a weight, and said waste product of said lignin biomass is less than 20% of said lignin biomass weight;
producing energy utilizing said lignin residues;
producing energy utilizing said waste product of said lignin biomass biomass;
recovering and recycling caustic from said processing of said lignin; and
using at least one product from said lignin biomass in the production of other derivative chemicals, materials, and products;
wherein choosing of a source of said lignin biomass provides a selective production of at least one of said products from said lignin biomass.
